SEQUENCE LISTING (54 sequences)

SEQ ID NO: 1 - protein, 9 aa, Homo sapiens
GLRILLLKV

SEQ ID NO: 2 - protein, 9 aa, Artificial Sequence
  Diastereomeric peptide
GLRILLLKV

SEQ ID NO: 3 - protein, 9 aa, Artificial Sequence
  enantiomeric peptide
GLRILLLKV

SEQ ID NO: 4 - protein, 12 aa, Mus musculus
GLRILLLKVAGF

SEQ ID NO: 5 - protein, 12 aa, Homo sapiens
GFRILLLKVAGF

SEQ ID NO: 6 - protein, 12 aa, Ovis aries
VFRILLLKVAGF

SEQ ID NO: 7 - protein, 13 aa, Gallus domesticus
LGLKIIFMKAVIF

SEQ ID NO: 8 - protein, 14 aa, Oncorhynchus mykiss
ILGLRILFLKTIVF

SEQ ID NO: 9 - protein, 14 aa, Ambystoma mexicanum
VLGLKIIFMKAVIF

SEQ ID NO: 10 - protein, 9 aa, Artificial Sequence
  Diastereomeric peptide
GFRILLLKV

SEQ ID NO: 11 - protein, 9 aa, Artificial Sequence
  Enantiomeric peptide
GFRILLLKV

SEQ ID NO: 12 - protein, 12 aa, Artificial Sequence
  Diastereomeric peptide
GLRILLLKVAGF

SEQ ID NO: 13 - protein, 12 aa, Artificial Sequence
  Diastereomeric peptide
GFRILLLKVAGF

SEQ ID NO: 14 - protein, 12 aa, Artificial Sequence
  Diastereomeric peptide
VFRILLLKVAGF

SEQ ID NO: 15 - protein, 13 aa, Artificial Sequence
  Diastereomeric peptide
LGLKIIFMKAVIF

SEQ ID NO: 16 - protein, 14 aa, Artificial Sequence
  Diastereomeric peptide
ILGLRILFLKTIVF

SEQ ID NO: 17 - protein, 14 aa, Artificial Sequence
  Diastereomeric peptide
VLGLKIIFMKAVIF

SEQ ID NO: 18 - protein, 27 aa, Homo sapiens
FQNLSVIGFRILLLKVAGFNLLMTLRL

SEQ ID NO: 19 - protein, 27 aa, Artificial Sequence
  Diastereomeric peptide
FQNLSVIGFRILLLKVAGFNLLMTLRL

SEQ ID NO: 20 - protein, 27 aa, Artificial Sequence
  Diastereomeric peptide
FQNLSVIGFRILLLKVAGFNLLMTLRL

SEQ ID NO: 21 - protein, 9 aa, Artificial Sequence
  Diastereomeric peptide
GLRILLLKV

SEQ ID NO: 22 - protein, 6 aa, Artificial Sequence
  Diastereomeric peptide
RILLLK

SEQ ID NO: 23 - protein, 6 aa, Artificial Sequence
  Diastereomeric peptide
RILLLK

SEQ ID NO: 24 - protein, 9 aa, Artificial Sequence
  Diastereomeric peptide
GLRILLLKV

SEQ ID NO: 25 - protein, 12 aa, Artificial Sequence
  Diastereomeric peptide
GFRILLLKVAGF

SEQ ID NO: 26 - protein, 12 aa, Artificial Sequence
  Diastereomeric peptide
GFRILLLKVAGF

SEQ ID NO: 27 - protein, 9 aa, Artificial Sequence
  Diastereomeric peptide
GLKILLLKV

SEQ ID NO: 28 - protein, 9 aa, Artificial Sequence
  Diastereomeric peptide
GLKILLLKV

SEQ ID NO: 29 - protein, 10 aa, Artificial Sequence
  Diastereomeric lipopeptide
XGLRILLLKV

SEQ ID NO: 30 - protein, 8 aa, Artificial Sequence
  Diastereomeric lipopeptide
XRILLLKX

SEQ ID NO: 31 - protein, 7 aa, Artificial Sequence
  Diastereomeric lipopeptide
XRILLLK

SEQ ID NO: 32 - protein, 10 aa, Artificial Sequence
  Diastereomeric lipopeptide
XGLRILLLKV

SEQ ID NO: 33 - protein, 13 aa, Artificial Sequence
  Diastereomeric lipopeptide
XGFRILLLKVAGF

SEQ ID NO: 34 - protein, 13 aa, Artificial Sequence
  Diastereomeric lipopeptide
XGFRILLLKVAGF

SEQ ID NO: 35 - protein, 10 aa, Artificial Sequence
  Diastereomeric lipopeptide
GLKILLLKVX

SEQ ID NO: 36 - protein, 8 aa, Artificial Sequence
  Diastereomeric lipopeptide
XRILLLKX

SEQ ID NO: 37 - protein, 6 aa, Artificial Sequence
  Diastereomeric peptide
RLLLLK

SEQ ID NO: 38 - protein, 9 aa, Artificial Sequence
  Diastereomeric peptide
GLRLLLLKV

SEQ ID NO: 39 - protein, 9 aa, Artificial Sequence
  Diastereomeric peptide
GLRILLAKV

SEQ ID NO: 40 - protein, 6 aa, Artificial Sequence
  Diastereomeric peptide
RILILK

SEQ ID NO: 41 - protein, 6 aa, Artificial Sequence
  Diastereomeric peptide
KLLLLK

SEQ ID NO: 42 - protein, 6 aa, Artificial Sequence
  Diastereomeric peptide
RILLLR

SEQ ID NO: 43 - protein, 9 aa, Artificial Sequence
  Diastereomeric peptide
GLKILLLKV

SEQ ID NO: 44 - protein, 12 aa, Artificial Sequence
  Diastereomeric peptide
GYRLLLLKVMGF

SEQ ID NO: 45 - protein, 12 aa, Artificial Sequence
  Diastereomeric peptide
GFRIMMLKVAGF

SEQ ID NO: 46 - protein, 9 aa, Artificial Sequence
  Diastereomeric peptide
GLKILLLKL

SEQ ID NO: 47 - protein, 10 aa, Artificial Sequence
  Diastereomeric peptide
GLKILLLKLL

SEQ ID NO: 48 - protein, 10 aa, Artificial Sequence
  Diastereomeric lipopeptide
XGLRILLLKL

SEQ ID NO: 49 - protein, 8 aa, Artificial Sequence
  Diastereomeric lipopeptide
XRLLLLKX

SEQ ID NO: 50 - protein, 7 aa, Artificial Sequence
  Diastereomeric lipopeptide
XRLLLLK

SEQ ID NO: 51 - protein, 9 aa, Artificial Sequence
  Synthetic peptide
GLGILLLGV

SEQ ID NO: 52 - protein, 9 aa, Artificial Sequence
  Synthetic peptide
GLRILLLKV

SEQ ID NO: 53 - protein, 9 aa, Artificial Sequence
  D-enantiomeric peptide
GLRILLLKV

SEQ ID NO: 54 - protein, 9 aa, Artificial Sequence
  Synthetic peptide
GLGILLLGV

(19) United States
(12) Patent Application Publication
(10) Pub. No.: US 2009/0275503 A1
(43) Pub. Date: Nov. 5, 2009

(54) DIASTEREOMERIC PEPTIDES FOR MODULATING T CELL IMMUNITY

(75) Inventors: Yechiel Shai, Yehud (IL); Irun R. Cohen, Rehovot (IL); Francisco Quintana, Buenos Aires (AR); Doron Gerber, Herzliya (IL)

Correspondence Address: FENNEMORE CRAIG, 3003 NORTH CENTRAL AVENUE, SUITE 2600, PHOENIX, AZ, 85012, US

(73) Assignee: Yeda Research and Developent Co., Ltd. at the Weizman Institute of Science, Rehovot (IL)

(21) Appl. No.: 12/066,626

(22) PCT Filed: Sep. 21, 2006
(86) PCT No.: PCT/IL06/01113

§ 371 Date: Oct. 6, 2008

Related U.S. Application Data
(60) Provisional application No. 60/719,169, filed on Sep. 22, 2005.

(51) Int. Cl. A61K 38/16; C07K 2/00; C07K 7/06; C07K 7/08; C07K 14/00; A61P 37/00; A61K 38/02; A61K 38/08; A61K 38/10

(52) U.S. Cl. 514/12; 530/300; 530/330; 530/329; 530/328; 530/327; 530/326; 530/325; 530/324; 514/2; 514/15; 514/14; 514/17; 514/16; 514/13

(57) ABSTRACT

The present invention provides diastereomeric peptides derived from the T Cell Receptor alpha Transmembrane Domain, and lipophilic conjugates thereof, which peptides and conjugates are effective in preventing or treating T cell mediated inflammatory diseases. The invention provides pharmaceutical compositions comprising these diastereomeric peptides and conjugates, and uses thereof for therapy of inflammatory diseases, autoimmunity and graft rejection.

### FIELD OF THE INVENTION

The present invention provides diastereomeric peptides and lipophilic conjugates thereof derived from the TCRα Transmembrane Domain, pharmaceutical compositions comprising same, and uses thereof for therapy of T cell mediated inflammatory diseases, autoimmunity and graft rejection.

### BACKGROUND OF THE INVENTION

T lymphocytes (T cells) are one of a variety of distinct cell types involved in an immune response. While the normal immune system is closely regulated, aberrations in immune responses are not uncommon. Numerous T cell-mediated inflammatory diseases are known, in which an inappropriate T cell response is a component of the disease. These include both diseases mediated directly by T cells, and also diseases in which an inappropriate T cell response contributes to the production of abnormal antibodies.

In some instances, the immune system functions inappropriately and reacts to a component of the host as if it were, in fact, foreign. Such a response results in an autoimmune disease, in which the host's immune system attacks the host's own tissue. T cells, as the primary regulators of the immune system, directly or indirectly affect such autoimmune pathologies. T cells also play a major role in the rejection for organ transplantation or graft versus host disease by bone marrow (hematopoietic stem cell) transplantation. Regulation of such immune responses is therefore therapeutically desired.

The activity of T cells is regulated by antigen, presented to a T cell in the context of a major histocompatibility complex (MHC) molecule. The T cell receptor (TCR) then binds to the MHC-antigen complex. Once antigen is complexed to MHC, the MHC-antigen complex is bound by a specific TCR on a T cell, thereby altering the activity of that T cell. The CD3/TCR is thus an attractive target for immunomodulation.

The CD3/T-cell receptor (TCR) complex of the majority of the mature T cells is a TCRαβ heterodimer associated to the γ, δ, ε and λ chains of CD3. This complex is stabilized by interactions between the transmembrane domain of the TCR chains and CD3 subunits. The interaction of the TCR with a peptide presented by the major histocompatibility complex molecule (MHC) induces a conformational change in the TCR that triggers CD3 phosphorylation.

A nine amino acid (aa) peptide derived from the transmembrane domain of the TCRα chain, denoted core peptide (CP), inhibits T-cell antigen specific activation in vitro and in vivo (Manolios et al., 1997). It was postulated that the CP peptide inhibits T-cell antigen specific activation by co-localizing with the TCR molecules, thereby inhibiting the proper assembly of the TCR-CD3 complex (Gerber et al., 2005; Manolios et al., 1997; Wang et al., 2002; Wang et al., 2002b; Bender et al., 2004). This interaction is thought to depend both on the secondary structure of CP and on the side chains of its two positive residues, since their substitution by glycines or a negatively charged aa abolishes the peptide's activity (Manolios et al., 1997; Bender et al., 2004). The present inventors have demonstrated recently that a mirror image peptide (all-D CP) can assemble with the TCR to the same extent as the wild-type (all-L CP) (Gerber et al., 2005). They hypothesized that this is due to structural reorientation of all-D CP that occurs within the membrane to accommodate for the change in helix chirality, which allows interactions with L molecules.

There are a number of disclosures using T Cell Receptor peptides as therapeutics for immune-related disease. For example, U.S. Pat. No. 5,614,192 discloses peptides capable of reducing the severity of a T cell mediated disease having an amino acid sequence comprising at least part of the second complementarity determining region of a T cell receptor characteristic of such T cell mediated disease.

WO 94/19470 discloses prophylactic and therapeutic compositions for the treatment of autoimmune diseases, which comprise a prophylactically or therapeutically effective amount of a soluble T-cell receptor α-chain produced by suppressor T-cells.

WO 97/43411 discloses polypeptides that contain substantially part or the whole of the constant region of a T-cell receptor α-chain, have immunosuppressive effects, but do not substantially cause any production of antibodies against themselves even when administered. This application discloses DNAs coding for the polypeptides as well as pharmaceutical compositions containing these polypeptides as the active ingredient.

U.S. Pat. No. 6,057,294 discloses peptides which affect T-cells, presumably by action on the T-cell antigen receptor, useful for therapy of inflammatory and autoimmune disease states involving the use of these peptides. The peptide is of the following formula: A-B-C-D-E in which: A is absent or 1 or 2 hydrophobic amino acids, B is a positively charged amino acid, C is a peptide consisting of 3 to 5 hydrophobic amino acids, D is a positively charged amino acid, and E is absent or up to 8 hydrophobic amino acids. The '294 patent does not disclose or suggest the use of peptides having D-isomeric amino acids.

Manolios et al. (1997) describe Conjugation of CP at the carboxyl terminus with palmitic acid via a Tris linker, which resulted in a greater inhibition of T-cell interleukin-2 (IL-2) production in vitro than peptide alone.

None of the background art discloses or suggests that CP-derived peptides having a severely perturbed secondary structure may retain their immunosuppressive activity. The background art does not disclose or suggest the production and use of lipophilic conjugates comprising fatty acids coupled to CP-derived diastereomeric peptides.

There exists a long-felt need for effective means of curing or ameliorating T cell mediated inflammatory or autoimmune diseases and ameliorating T cell mediated pathologies. Traditional reagents and methods used to attempt to regulate an immune response in a patient also result in unwanted side effects and have limited effectiveness. For example, immunosuppressive reagents (e.g., cyclosporin A, azathioprine, and prednisone) used to treat patients with autoimmune diseases also suppress the patient's entire immune response, thereby increasing the risk of infection, and can cause toxic side effects to non-lymphoid tissues. In addition, usually only the symptoms of the disease can be treated, while the disease continues to progress, often resulting in severe debilitation or death. Such a treatment should therefore ideally control the inappropriate T cell response, rather than merely reducing the symptoms.

### SUMMARY OF THE INVENTION

The present invention provides diastereomeric peptides and lipopeptides derived from the T cell receptor alpha (TCRα) Transmembrane Domain (TM), pharmaceutical compositions comprising same, and uses thereof for therapy of T cell mediated inflammatory diseases, autoimmunity and graft rejection.

Unexpectedly, it is now disclosed for the first time that disruption of the secondary structure of the known peptide derived from TCRα TM, denoted Core Peptide (CP) does not abolish the peptide's immunosuppressive activity. The invention discloses for the first time that diastereomeric CP incorporating both D and L amino acids, wherein the resulting peptide may optionally be conjugated to fatty acids, can unexpectedly endow the peptide with superior immunosuppressive activities Compared to the native CP.

The present invention provides diastereomeric peptides, derivatives and conjugates thereof, having an amino acid sequence based on a fragment of the TCRα TM. In one embodiment, the fragment is a peptide derived from murine TCRα TM, herein denoted CP, having the amino acid sequence GLRILLLKV (SEQ ID NO: 1). In other embodiments, the peptide is derived from the TCRα TM of other species, e.g. mammals, birds, reptiles, fish and amphibians. Certain non-limiting examples of homologous TCRα TM fragments of selected species, denoted as SEQ ID NOS:4-9, are presented in Table 1 hereinbelow.

According to a first aspect, there is provided a diastereomeric peptide derived from a TCR alpha chain transmembrane domain, the peptide comprising at least two basic amino acid residues.

The term “diastereomeric peptide” denotes peptides having both D-amino acid residues and L-amino acid residues. The location of the D-amino acid residues may vary so long as the inhibitory activity of the peptide on T cell activation is retained. In one particular embodiment, the peptide comprises at least two D-amino acid residues.

In one embodiment, the peptide comprises an amino acid sequence as set forth in SEQ ID NO: 1 wherein at least one amino acid residue is of the D-isomer configuration.

In another embodiment, the diastereomeric peptide is 2D-CP, having an amino acid sequence as set forth in SEQ ID NO:2 (the D-amino acid residues are bold and underlined):

GLRILLLKV.

According to various embodiments of the present invention, there are provided diastereomeric peptide derivatives, fragments, analogs, extensions, conjugates and salts of CP and homologs thereof, wherein said peptides comprise at least two basic amino acid residues and are not known proteins or peptides. In certain embodiments, the two basic amino acid residues may be separated by 3-5 hydrophobic (non-polar) amino acid residues. In one particular embodiment, the two basic amino acid residues are separated by four hydrophobic amino acid residues. The amino acid sequences of certain non-limiting examples of such CP-derived diastereomeric peptides are presented in Table 2 hereinbelow.

The peptides, derivatives, fragments, analogs, extensions and salt thereof according to the invention are preferably from 5 to 50 amino acids in length, more preferably from 5 to 30 amino acids in length, and most preferably from 7 to 15 amino acids in length.

In another particular embodiment, said diastereomeric peptide is derived from human TCRα TM. In certain other particular embodiments, said peptide has an amino acid sequence as set forth in SEQ ID NO:10 (GFRILLLKV; the D-amino acid residues are bold and underlined) or derivatives, fragments, analogs, extensions, conjugates and salts thereof.

According to certain other particular embodiments, the diastereomeric peptide has an amino acid sequence as set forth in any one of SEQ ID NOS:12-17 as set forth in Table 2 below. In other particular embodiments, the diastereomeric peptide has an amino acid sequence as set forth in any one of SEQ ID NOS:19-28. In yet another particular embodiment, the diastereomeric peptide is a CP analog or derivative having an amino acid sequence as set forth in any one of SEQ ID NOS:37-47 (see Table 2).

In another embodiment, the diastereomeric peptide is conjugated to a lipophilic moiety.

According to one embodiment of the invention, the lipophilic moiety is a fatty acid. In one embodiment, the fatty acid is selected from the group consisting of saturated, unsaturated, monounsaturated, polyunsaturated and branched fatty acids. According to currently preferred embodiments, the fatty acids consist of at least three, preferably at least six, and more preferably at least eight carbon atoms. Examples of the fatty acids that may be coupled to the peptides of the invention include, but are not limited to, octanoic acid (OA), decanoic acid (DA), undecanoic acid (UA), dodecanoic acid (DDA; lauric acid), myristic acid (MA), palmitic acid (PA), stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid. In a preferred embodiment, the fatty acid is octanoic acid. According to certain other currently preferred embodiments, the fatty acid is selected from decanoic acid, undecanoic acid, dodecanoic acid, myristic acid, and palmitic acid.

The fatty acid may be coupled to the N-terminus of the peptide, to the C-terminus, or to any other free functional group along the peptide chain, for example, to the ε-amino group of lysine.

In one particular embodiment, the diastereomeric peptide lipophilic conjugate (lipopeptide) has an amino acid sequence as set forth in SEQ ID NO:29, presented in Table 2 below. In other particular embodiments, the diastereomeric lipopeptide has an amino acid sequence as set forth in any one of SEQ ID NOS:30-36. In certain other particular embodiments, the diastereomeric lipopeptide has an amino acid sequence as set forth in any one of SEQ ID NOS:48-50 (see Table 2).

In another aspect, there is provided a peptide derived from a T cell receptor (TCR) alpha chain transmembrane domain, the peptide comprising at least two basic amino acid residues, wherein all amino acid residues of said peptide are of the “D” isomer configuration.

In another aspect, the invention provides an enantiomer peptide having an amino acid sequence as set forth in any one of SEQ ID NOS:3 (GLRILLLKV, denoted all-D CP; D-amino acid residues are bold and underlined) and 11 (GFRILLLKV, denoted human all-D CP; D-amino acid residues are bold and underlined). In other embodiments, the invention provides lipophilic conjugates comprising a peptide having an amino acid sequence as set forth in any one of SEQ ID NOS:3 and 11 coupled to a fatty acid.

The diastereomeric and enantiomeric peptides and conjugates of the present invention are effective in many T-cell mediated pathologies, including, but not limited to: multiple sclerosis, rheumatoid arthritis, juvenile rheumatoid arthritis, autoimmune neuritis, systemic lupus erythematosus, psoriasis, Type I diabetes, Sjogren's disease, thyroid disease, myasthenia gravis, sarcoidosis, autoimmune uveitis, inflammatory bowel disease (Crohn's and ulcerative colitis) autoimmune hepatitis, idiopathic thrombocytopenia, scleroderma, alopecia areata, hemolytic anemia, glomerulonephritis, dermatitis and pemphigus, T-cell mediated inflammatory diseases, allergies and graft rejection.

In another aspect, the invention provides pharmaceutical compositions comprising as an active ingredient a peptide or conjugate of the invention, and a pharmaceutically acceptable carrier, excipient or diluent.

In other aspects, the invention provides methods of treating or preventing a T-cell mediated pathology in a subject in need thereof, comprising administering to the subject a therapeutically effective amount of a peptide or conjugate of the invention.

In one embodiment, the T cell mediated pathology is an autoimmune disease. In another embodiment the autoimmune disease is selected from the group consisting of: multiple sclerosis, rheumatoid arthritis, juvenile rheumatoid arthritis, autoimmune neuritis, systemic lupus erythematosus, psoriasis, Type I diabetes, Sjogren's disease, thyroid disease, myasthenia gravis, sarcoidosis, autoimmune uveitis, inflammatory bowel disease (Crohn's and ulcerative colitis), autoimmune hepatitis, idiopathic thrombocytopenia, scleroderma, alopecia areata, hemolytic anemia, glomerulonephritis, dermatitis and pemphigus. In a particular embodiment, the autoimmune disease is rheumatoid arthritis.

In another embodiment, the T cell mediated pathology is a T cell mediated inflammatory or allergic disease. In a particular embodiment, the inflammatory or allergic disease is delayed type hypersensitivity.

In another embodiment, the T cell mediated pathology is graft rejection.

In another aspect, the invention provides a method of inhibiting T-cell activation in a subject in need thereof, comprising administering to the subject a therapeutically effective amount of a peptide or conjugate of the invention.

These and other embodiments of the present invention will be better understood in relation to the description, figures, examples, and claims that follow.

### BRIEF DESCRIPTION OF THE DRAWINGS

FIG. 1. 2D-CP does not fold into an α-helical structure. A. CD spectra of 2D-CP in H₂O (black diamonds), 2D-CP in 1% LPC micelles (empty diamonds) and all-L CP in 1% LPC micelles (empty squares). B. Structures generated for all-L CP (left) and 2D-CP (right) by a molecular dynamic simulation, showing that D aa disturb the α-helical structure of CP. C. All Atom RMSD profile across the first 5 ns of the simulation of all-L CP in the lipid bilayer. D. The RMSD profile of 2D-CP.

FIG. 2. The 2D-CP peptide co-localizes with the TCR receptor. A. The TCR is visualized using a TCR-FITC antibody. Excitation was at 488 nm and the emission was collected between 505 and 525 nm. B. The 2D-CP was visualized using a Rhodamine probe attached to the N-terminus. Excitation was at 543 nm and emission was collected from 560 nm and up. C. Merging of the two channels before bleach demonstrates that the 2D-CP co-localizes with the TCR. D. The circle surrounds the area which underwent bleaching. Bleach was achieved with the laser (543 nm) set at 100% power for 6 seconds. Thus, the FITC donor molecule was not affected. The increase in green demonstrates that there was fluorescence energy transfer between the Rhodamine-labeled 2D-CP peptide and the TCR-FITC.

FIG. 3. The 2D-CP peptide interferes with T-cell activation. LNC from Mt primed rats were activated in vitro with PPD (A) or with Mt176-90 (B), in the presence of wild-type all-L CP (black), 2D-CP (gray) or 2G CP (white).

FIG. 4. Inhibition of adjuvant arthritis (AA) by wild-type and 2D-CP. Adjuvant Arthritis was induced with Mt in oil, mixed with all-L CP (wild-type), 2D-CP, 2G CP or PBS. Arthritis was scored every 2-3 days, starting at day 10. A. Time course of the AA score, presented as the mean±SEM. p<0.05 when the results of 2D-CP or all-L CP treated animals are compared with those of the control groups treated with PBS or 2G CP. B. Paw swelling measured at day 26 after AA induction. The results are presented as the mean±SEM of the difference between the values for hind limb diameter taken at days 0 and 26. p<0.05 between the control groups treated with PBS or 20 CP and all-L or 2D-CP treated rats (p<0.05). C. Dose dependency of the effect of the 2D-CP on AA, as measured by paw swelling at day 26. D. DTH was measured as described in the methods section to quantify the effect of the peptides on the immune response to Mt. The results are expressed as percent of the uninhibited DTH response to Mt. The reduction observed on rats treated with all-L CP or 2D-CP is significant (p<0.05) when compared to the effect of 2G CP.

FIG. 5. The 2D-CP inhibits DTH response to oxazalone in mice. DTH response measured 1 hr after challenge with oxazalone of mice treated with DMSO alone, all-L CP, 2D-CP or dexamethasone. The results are expressed as the decrease in ear thickness obtained after treatment with the peptides, relative to the decrease observed in dexamethasone-treated mice (which was considered 100%), ±SEM.

FIG. 6. Both CP and 2D-CP co-immunoprecipitate together with the TCR. CP, 2D-CP or 2G-CP were incubated either together with activated A2B cells and either the (a) TCR or (b) MHC 1 molecules were immunoprecipitated. CP and 2D-CP co-immunoprecipitated together with the TCR significantly more than the 2G-CP mutant. The peptides did not co-immunoprecipitate together with the MHC 1 molecule to any significant extent.

FIG. 7. The structural difference between L and D-CP is illustrated. The Arginine and Lysine side chains are visible in order to demonstrate the effect of the D-amino acid substitutions on side-chain location. The backbone of D-CP turns in the opposite direction than the wild type analogue.

FIG. 8. The L and D-CP have mirror image structures. Far-UV Circular Dichroism spectra of L-CP (triangles) and D-CP (diamonds) were collected in a membrane mimetic environment (1% LPC). Spectra were measured on an Aviv spectropolarimeter at 1 nm intervals with 20 sec averaging time, using a 0.1 cm light path. The Y axis represents raw data (mdeg) after subtracting the background spectrum of 1% LPC alone. The structures are not canonical α-helices as can be expected for such short peptides (a population with random coil conformation is likely). However, the spectra of the L-CP and D-CP are exactly mirror image.

FIG. 9. Both L and D-CP inactivate T cells in vitro in a similar fashion. Inhibition of T cell activation was measured by following proliferation after antigen specific activation. T cells were activated with PPD (A) or with Mt176-90 (B). The activation was in the presence of L-CP (black), D-CP (gray) or 2G CP (white) at concentrations of 1 μg/ml, 5 μg/ml and 75 μg/ml.

FIG. 10. Inhibition of AA by L and D-CP. AA was induced by immunization to Mt in oil, mixed with L-CP, D-CP, 2G CP or PBS (3 rats per group). Arthritis was scored every 2-3 days, starting at day 10. Panel A demonstrates the time course of the AA disease. Panel B presents leg swelling scores measured at day 26 after AA induction. The results are presented as the mean±SEM of the difference between the values for hind limb diameter taken at days 0 and 26. The presence of both L and D-CP significantly reduces the severity of AA compared with the control groups (p<0.05).

FIG. 11. The L-CP and D-CP affect cellular immune responses in vivo. Rats were immunized with Mt to induce AA in the presence of L-CP, D-CP, 2G CP, or PBS. DTH was measured as described in the methods section to quantify the effect of the peptides on the immune response to Mt. The results were normalized as percent inhibition of the DTH response. The values measured for rats co-immunized with PBS were considered as zero inhibition. The results demonstrate a significant effect in vivo for the L and D-CP.

FIG. 12. The L and D-CP peptides colocalizes with the TCR receptor in the membrane. (A) The TCR is visualized using αTCR-FITC. Excitation was at 488 nm and the emission was collected between 505 and 525 nm. (B) The peptides are visualized using a Rhodamine probe attached to their N-terminus. Excitation was at 543 nm and emission was collected from 560 nm and up. (C) Merging of (A) and (B) demonstrates that all the CP peptides co-localize with TCR. (D) Point bleach at 543 nm, with the laser at 100% for 6 sec, demonstrates that there is energy transfer between the CP-Rho peptides and the TCR FITC-labeled antibody. The arrows points to the area which underwent the bleaching procedure.

### DETAILED DESCRIPTION OF THE INVENTION

The present invention provides diastereomeric peptides derived from the TCRα Transmembrane Domain Core Peptide (CP), and lipophilic conjugates thereof, which peptides and conjugates are effective in preventing or treating T cell mediated inflammatory diseases. The invention provides pharmaceutical compositions comprising these diastereomeric peptides and conjugates, and uses thereof for therapy of inflammatory diseases, autoimmunity and graft rejection.

The use of diastereomeric peptides has been described in the art (see, e.g. WO 2005/060350 and US 2004/053847).

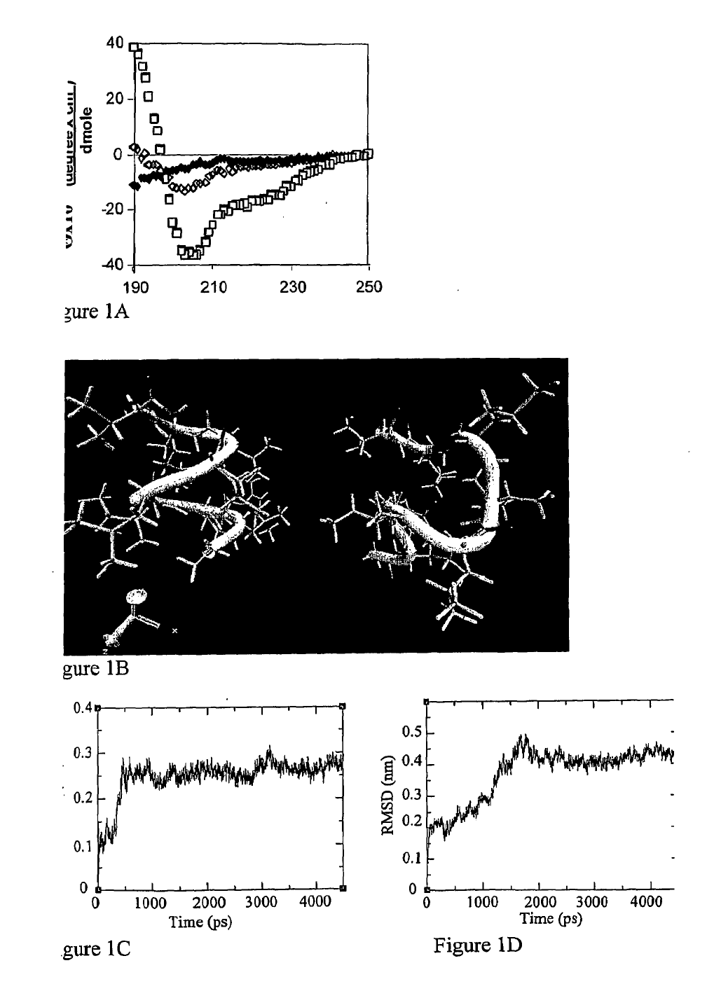

Unexpectedly, it is now disclosed that diastereomeric peptides derived from CP retain the immunosuppressive activity of the native peptide, despite the observation that the two D aa introduced in CP perturb its secondary structure (FIG. 1).

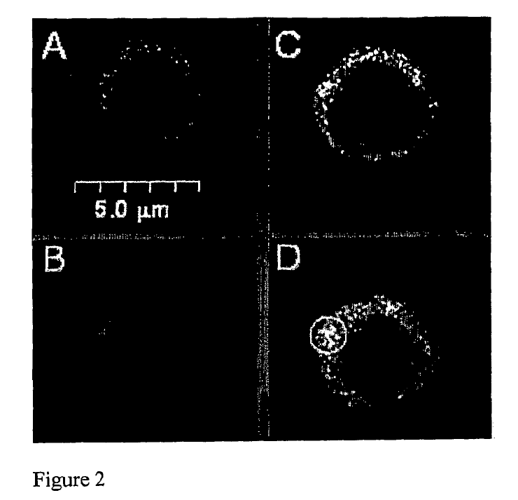

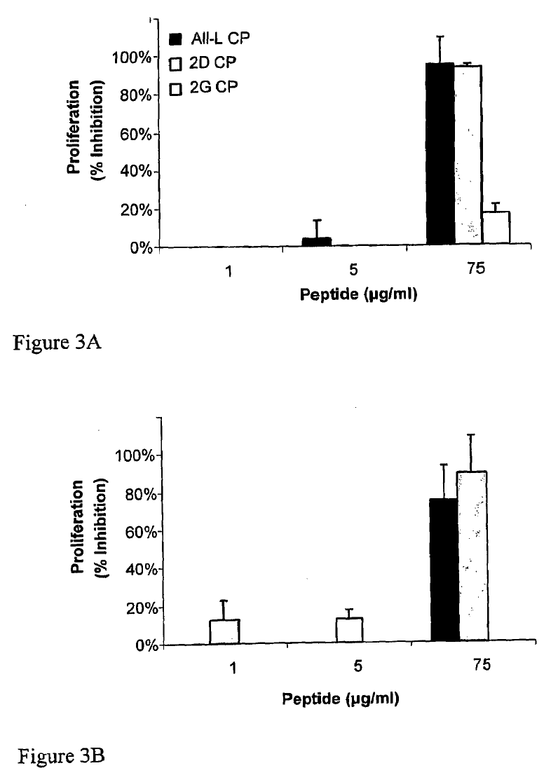

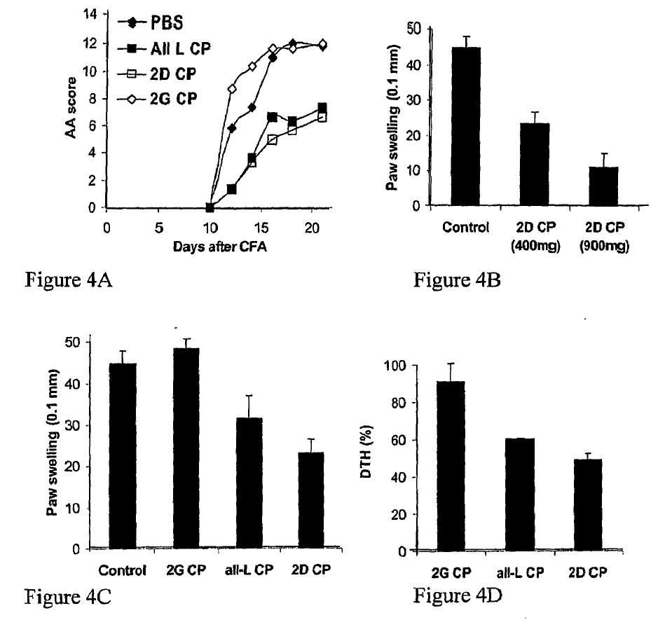

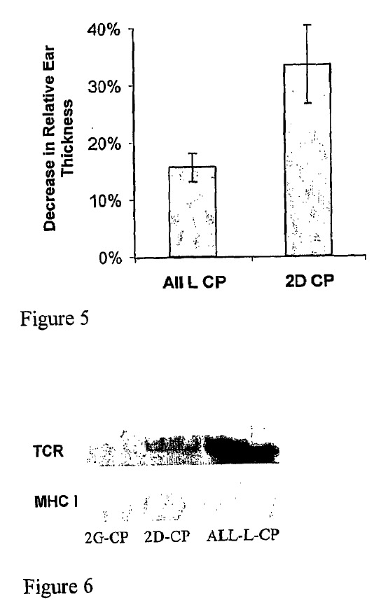

The present invention is based, in part, on the surprising finding that, upon binding to the membrane, a diastereomeric peptide termed 2D-CP (SEQ ID NO:2) displays wild-type functionality: it co-localizes with the TCR complex (FIG. 2) and interferes with antigen-triggered T cell activation (FIGS. 3, 4 and 5). Remarkably, the diastereomer peptide 2D-CP showed a stronger immunosuppressive activity than did wild-type all-L CP. In vitro, 2D-CP was more active at lower concentrations than all-L CP (FIG. 3). In vivo, the administration of 2D-CP led to a greater reduction in the clinical signs of AA when compared to the all-L CP (FIG. 4), and was twice more effective than all-L CP when used to inhibit a DTH response in a therapeutic setting (FIG. 5).

Peptides, Derivatives and Conjugates

The peptides of the invention may be synthesized or prepared by techniques well known in the art. The peptides can be synthesized by a solid phase peptide synthesis method of Merrifield (1963). Alternatively, a diastereomeric peptide of the present invention can be synthesized using standard solution methods well known in the art (see, for example, Bodanszky, 1984) or by any other method known in the art for peptide synthesis.

The present invention provides peptide derivatives and conjugates thereof, having an amino acid sequence based on a fragment of the TCRα Transmembrane Domain, herein denoted Core Peptide (CP): GLRILLLKV (SEQ ID NO:1). Unless otherwise specified, the amino acid residues described herein may either be in the “L” isomeric form or in the “D” isomeric form.

Specifically, the invention provides diastereomeric peptides derived from T cell receptor alpha chain (TCRα) transmembrane domain (TM), the peptides comprising at least two basic amino acid residues.

The term “derived from” refers to construction of a peptide based on the knowledge of a sequence using any one of the suitable means known to one skilled in the art, e.g. chemical synthesis in accordance with standard protocols in the art. A peptide derived from TcRα TM sequence can be an analog, fragment, conjugate or derivative of a native TcRα TM sequence, and salts thereof, as long as the peptide comprises at least two positively-charged amino acids, and said peptide retains its ability to inhibit T cell activation. The synthetic diastereomeric peptides of the invention comprise both L amino acids and D isomers of natural occurring L amino acids, and may comprise other artificial amino acids (amino acid mimetics) or non-natural amino acids. TcRα TM sequences typically comprise two positively-charged amino acids which are separated by a hydrophobic sequence. Advantageously, the TcRα TM-derived peptides of the invention comprise two positively-charged amino acids which are separated by 3-5, or in another embodiment, 4 hydrophobic amino acid residues.

The amino acid sequences included in the transmembrane domain of a TCRα chain may be determined readily by those of ordinary skill in the art, using e.g. transmembrane domain prediction algorithms. TcRα TM sequences are typically between about 20-30 amino acids in length. For example, human TcRα TM, denoted by SEQ ID NO:18, is presented in Table 2 hereinbelow.

Hydrophobicity is generally defined with respect to the partition of an amino acid between a nonpolar solvent and water. Hydrophobic amino acids are those acids which show a preference for the nonpolar solvent. Examples of naturally occurring hydrophobic amino acids are aliphatic amino acids alanine, isoleucine, leucine, methionine, proline, and valine, and aromatic amino acids tryptophan and phenylalanine. These amino acids confer hydrophobicity as a function of the length of aliphatic and size of aromatic side chains when found as residues within a protein. Hydrophobic amino acids also include amino acids that are not encoded by the genetic code, e.g. α-aminoisobutyric acid.

According to one embodiment, the invention provides a diastereomeric peptide comprising an amino acid sequence as set forth in SEQ ID NO:1, wherein at least one amino acid residue, or in another embodiment at least two amino acid residues of the diastereomeric peptide are of the D-isomer configuration.

In another embodiment, the diastereomeric peptide is 2D-CP, having an amino acid sequence as set forth in SEQ ID NO:2-GLRILLLKV (the bold and underlined amino acid residues at positions 3 and 8 are of the “D” isomer configuration). In other embodiments, derivatives, fragments, analogs, extensions, conjugates and salts thereof are contemplated, with the proviso that the peptide or derivative is not a known protein or peptide, as detailed below.

The peptides of the invention are preferably from 5 to 50 amino acids, more preferably from 5 to 30 amino acids, and most preferably from 7 to 15 amino acids. It should be understood that a diastereomeric peptide of the invention need not be identical to the amino acid sequence of SEQ ID NO:2 so long as its immunosuppressive activity is retained, and preferably increased, as described herein.

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, the substitution between glycine and serine, the substitution of one basic residue such as lysine, arginine or histidine for another, or the substitution of one acidic residue, such as aspartic acid or glutamic acid for another.

A peptide derivative refers to a molecule comprising the amino acid sequence of a peptide of the invention subject to various changes, including, but not limited to, chemical modifications, substitutions, insertions, extensions and deletions where such changes do not destroy the immunosuppressive activity of the peptide, and such derivative is not a known peptide or protein.

Peptide derivatives having chemical modifications include, for example, any chemical derivative of the peptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine. According to one particular embodiment, the peptide is amidated at its C-terminus.

Peptide derivatives of the invention are constructed such that they are substantially identical to the sequence from which they are derived. Preferably, the diastereomeric peptides of the invention may have at least about 40% identity in their amino acid sequence, more preferably at least about 50%, more preferably at least about 70% and most preferably at least about 90% identity to the amino acid sequence of 2D-CP (SEQ ID NO:2). It should be understood that peptide derivatives of the invention comprise the consensus sequence of two basic amino acid residues that are separated by a hydrophobic amino acid sequence, preferably by 3-5 hydrophobic amino acids.

Peptides of the present invention also include any peptide having one or more additions and/or deletions of residues relative to the sequence of the peptides of the invention, the sequences of which are shown herein, so long as the requisite inhibitory activity on T cell activation is maintained. The term “fragment” or “active fragment” thus relates to a peptide portion of a full length peptide of the invention (e.g. 2D-CP) that retains the at least two positively charged amino acids and has at least one activity that is characteristic of the corresponding full-length peptide. Thus, for example, a fragment of the TCRα TM comprises an amino acid sequence identical to a portion of the TM, but excluding the full-length TM. Amino acid extensions may consist of a single amino acid residue or stretches of residues. The extensions may be made at the carboxy or amino terminal end of a diastereomeric peptide, as well as at a position internal to the peptide. Such extensions will generally range from 2 to 15 amino acids in length, wherein the distance between the two positively-charged amino acid residues is preferably no longer than five amino acids in length. In certain embodiments, the diastereomeric peptide may comprise a derivative of the full-length TCRα Transmembrane Domain or an active fragment thereof (see, e.g., SEQ ID NOS:18 and 4-9 in Tables 2 and 1, respectively), with the proviso that it is not a known protein or peptide. In certain particular embodiments, the diastereomeric peptide comprises a sequence corresponding to the transmembrane domain of a TCRα chain, wherein at least one amino acid residue is of the “D” isomer configuration, but lacks other regions of the TCRα chain, e.g. the cytoplasmic region, the extracellular region or a substantial portion thereof.

According to certain particular embodiments, the diastereomeric peptide has an amino acid sequence as set forth in any one of SEQ ID NOS:12-17, presented in Table 2 below. In other particular embodiments, the diastereomeric peptide has an amino acid sequence as set forth in any one of SEQ ID NOS:19-28 (see Table 2). In yet another particular embodiment, the diastereomeric peptide is a CP analog or derivative having an amino acid sequence as set forth in any one of SEQ ID NOS:37-47 (see Table 2).

A diastereomeric peptide of the present invention may be coupled to or conjugated with another protein or polypeptide to produce a conjugate. Such a conjugate may have advantages over the peptide used alone. For example, a diastereomeric peptide of the invention may be conjugated to an antigen involved in a T cell mediated pathology. Without wishing to be bound by any theory or mechanism of action, vaccination with such a conjugate may result in reduced T cell activation to the conjugated antigen, and thereby induce a tolerogenic immune response to said disease target antigen, or alternatively alter the cytokine profile of T cells responding to said antigen (e.g. from Th1 to Th2). The peptides can be conjugated directly via an amide bond, synthesized as a dual ligand peptide, or joined by means of a linker moiety as is well known in the art to which the present invention pertains.

Examples of known antigens involved in autoimmune diseases include but are not limited to myelin basic protein, myelin oligodendrocyte glycoprotein and myelin proteolipid protein (involved in multiple sclerosis), acetylcholine receptor components (involved in myasthenia gravis), collagen and Mycobacterial hsp peptide 180-188 (involved in arthritis), laminin and p53 peptide (involved in systemic lupus erythematosis) and Ags involved in insulin-dependent diabetes such as p277 (positions 437-460 of human HSP60) and glutamic acid decarboxylase (GAD).

In another embodiment, the invention provides a lipophilic conjugate comprising a diastereomeric peptide coupled to a fatty acid, the diastereomeric peptide comprising an amino acid sequence as set forth in SEQ ID NO:1 wherein at least one amino acid residue of the diastereomeric peptide is of the D-isomer configuration, and analogs, fragments, derivatives and extensions thereof. In one embodiment, at least two amino acid residues of the diastereomeric peptide are of the D-isomer configuration.

The terms “lipophilic conjugate” and “lipopeptide” used interchangeably throughout the specification and claims designate a conjugate comprising a peptide covalently coupled to a lipophilic moiety, e.g. a fatty acid.

The fatty acid that can be coupled to the peptides of the invention is selected from saturated, unsaturated, monounsaturated, polyunsaturated and branched fatty acids. Typically, the fatty acid consists of at least three, preferably at least six, and more preferably at least eight carbon atoms, such as, for example, octanoic acid (OA) decanoic acid (DA), undecanoic acid (IA), dodecanoic acid (laulic acid), myristic acid (MA), palmitic acid (PA), stearic acid, arachidic acid, lignoceric acid, palmitoleic acid, oleic acid, linolenic acid, arachidonic acid, trans-hexadecanoic acid, elaidic acid, lactobacillic acid, tuberculostearic acid, and cerebronic acid. In one embodiment, the lipophilic moiety is an ocyl group, In another particular embodirnent, said fatty acid is selected from decanoic acid, undecanoic acid, dodecanoic acid and myristic acid.

The fatty acid may be coupled to the N-terminus, to the C-terminus, or to any other free functional group along the peptide chain, for example, to the ε-amino group of lysine. Coupling of a fatty acid to a peptide is performed similarly to the coupling of an amino acid to a peptide during peptide synthesis. It should be understood that the fatty acid is covalently coupled to the peptide. The terms “coupling” and “conjugation” are used herein interchangeably and refer to the chemical reaction, which results in covalent attachment of a fatty acid to a peptide to yield a lipophilic conjugate.

In one particular embodiment, the lipophilic moiety is conjugated to the peptide directly. In another particular embodiment, the lipophilic moiety is conjugated to the peptide via a linker.

In one particular embodiment, the diastereomeric peptide is a lipopeptide having an amino acid sequence as set forth in SEQ ID NO:29, or, in other embodiments, 30-36 (Table 2). In other particular embodiments, the diastereomeric peptide is a CP analog or derivative lipopeptide having an amino acid sequence as set forth in any one of SEQ ID NOS:48-50 (Table 2).

The peptides of the invention may be derived from the TM of murine TcRα or homologs thereof, i.e. sequences that are significantly related thereto because of an evolutionary relationship between species. Table 1 presents wild-type TcRα transmembrane domain (TM) fragments of various species. The highly conserved basic amino acids are in bold italic font.

TABLE 1
TcRα TM sequences:

| SEQ | | | |
|---|---|---|---|
| ID | | | |
| NO: | Source | Sequence | Reference |
| 4 | Mouse | GLRILLLKVAGF | Enk et al., 2000 |
| 4 | Rat | GLRILLLKVAGF | Enk et al., 2000 |
| 5 | Human | GFRILLLKVAGF | Enk et al., 2000; |
| | | | Manolios et al., 1990 |
| 6 | Sheep | VFRILLLKVAGF | Enk et al., 2000 |
| 6 | Cow | VFRILLLKVAGF | Enk et al., 2000 |
| 7 | Chicken | LGLKIIFMKAVIF | Gobel et al., 1994 |
| 8 | Trout | ILGLRILFLKTIVF | Partula et al., 1996 |
| 9 | Axolotl | VLGLKIIFMKAVIF | Partula et al., 1996 |
| | (Ambystoma | | |
| | mexicanum) | | |

In another particular embodiment, said diastereomeric peptide is derived from human TCRα TM. In certain other particular embodiments, said peptide has an amino acid sequence as set forth in SEQ ID NO:10 (GFRILLLKV; the D-amino acid residues are bold and underlined) or derivatives, fragments, analogs, extensions, conjugates and salts thereof.

Table 2 presents TcRα transmembrane domain (TM)-derived peptides, including exemplary diastereomeric peptides and lipopeptides of the invention as well as native (all-L) and enantiomeric (all-D) TM-derived sequences described herein. D-amino acid residues are bold and underlined.

TABLE 2
TcRα TM derived peptides.

| SEQ | | |
|---|---|---|
| ID | | |
| NO: | Description | Sequence |
| 1 | CP (core peptide) | GLRILLLKV |
| 2 | 2D-CP (CP | GLRILLLKV |
| | diastereomeric | |
| | peptide) | |
| 3 | D-CP (all-D CP) | GLRILLLKV |
| 10 | h2D-CP (human CP | GFRILLLKV |
| | diastereomeric | |
| | peptide) | |
| 11 | hD-CP (human all-D | GFRILLLKV |
| | CP) | |
| 12 | Mouse TcRα TM- | GLRILLLKVAGF |
| | derived | |
| | diastereomeric | |
| | peptide | |
| 13 | Human TcRα TM- | GFRILLLKVAGF |
| | derived | |
| | diastereomeric | |
| | peptide | |
| 14 | Sheep TcRα TM- | VFRILLLKVAGF |
| | derived | |
| | diastereomeric | |
| | peptide | |
| 15 | Chicken TcRα TM- | LGLKIIFMKAVIF |
| | derived | |
| | diastereomeric | |
| | peptide | |
| 16 | Trout TcRα TM- | ILGLRILFLKTIVF |
| | derived | |
| | diastereomeric | |
| | peptide | |
| 17 | Axolotl TcRα TM- | VLGLKIIFMKAVIF |
| | derived | |
| | diastereomeric | |
| | peptide | |
| 18 | Human TcRα TM | FQNLSVIGFRILLLKVAGFNLLMTLRL |
| 19 | Human TcRa TM- | FQNLSVIGFRILLLKVAGFNLLMTLRL |
| | derived | |
| | diastereomeric | |
| | peptide | |
| 20 | Human TcRα TM- | FQNLSVIGFRILLLKVAGFNLLMTLRL |
| | derived | |
| | diastereomeric | |
| | peptide | |
| 21 | CP-derived | GLRILLLKV |
| | diastereomeric | |
| | peptide | |
| 22 | CP-derived | RILLLK |
| | diastereomeric | |
| | peptide | |
| 23 | CP-derived | RILLLK |
| | diastereomeric | |
| | peptide | |
| 24 | CP-derived | GLRILLLKV |
| | diastereomeric | |
| | peptide | |
| 25 | CP-derived | GFRILLLKVAGF |
| | diastereomeric | |
| | peptide | |
| 26 | CP-derived | GFRILLLKVAGF |
| | diastereomeric | |
| | peptide | |
| 27 | CP-derived | GLKILLLKV |
| | diastereomeric | |
| | peptide | |
| 28 | CP-derived | GLKILLLKV |
| | diastereomeric | |
| | peptide | |
| 29 | CP-derived | Octyl-GLRILLLKV |
| | diastereomeric | |
| | lipopeptide | |
| 30 | CP-derived | Octyl-RILLLK-Octyl |
| | diastereomeric | |
| | lipopeptide | |
| 31 | CP-derived | Decyl-RILLLK |
| | diastereomeric | |
| | lipopeptide | |
| 32 | CP-derived | Octyl-GLRILLLKV |
| | diastereomeric | |
| | lipopeptide | |
| 33 | CP-derived | Decyl-GFRILLLKVAGF |
| | diastereomeric | |
| | lipopeptide | |
| 34 | CP-derived | Undecyl-GFRILLLKVAGF |
| | diastereomeric | |
| | lipopeptide | |
| 35 | CP-derived | GLKILLLKV-Decyl |
| | diastereomeric | |
| | conjugate | |
| 36 | CP-derived | Decyl-RILLLK-Decyl |
| | diastereomeric | |
| | lipopeptide | |
| 37 | CP-derived | RLLLLK |
| | diastereomeric | |
| | peptide | |
| 38 | CP-derived | GLRLLLLKV |
| | diastereomeric | |
| | peptide | |
| 39 | CP-derived | GLRILLAKV |
| | diastereomeric | |
| | peptide | |
| 40 | CP-derived | RILILK |
| | diastereomeric | |
| | peptide | |
| 41 | CP-derived | KLLLLK |
| | diastereomeric | |
| | peptide | |
| 42 | CP-derived | RILLLR |
| | diastereomeric | |
| | peptide | |
| 43 | CP-derived | GLKILLLKV |
| | diastereomeric | |
| | peptide | |
| 44 | CP-derived | GYRLLLLKVMGF |
| | diastereomeric | |
| | peptide | |
| 45 | CP-derived | GFRIMMLKVAGF |
| | diastereomeric | |
| | peptide | |
| 46 | CP-derived | GLKILLLKL |
| | diastereomeric | |
| | peptide | |
| 47 | CP-derived | GLKILLLKLL |
| | diastereomeric | |
| | peptide | |
| 48 | CP-derived | Octyl-GLRILLLKL |
| | diastereomeric | |
| | lipopeptide | |
| 49 | CP-derived | Octyl-RLLLLK-Octyl |
| | diastereomeric | |
| | peptide | |
| 50 | CP-derived | Decyl-RLLLLK |
| | diastereomeric | |
| | peptide | |

It should be understood that the fatty acid may be varied and the lipopeptides disclosed are non-limitative exemplary embodiments.

Intermolecular interactions are sterically constrained; accordingly no sequence-specific interactions were thought to occur between D and L-stereoisomers. Surprisingly, it is disclosed that a D-stereoisomer of CP (D-CP) is able to inhibit T-cell activation. L-CP and D-CP co-localized with the TCR in the membrane and inhibited T-cell activation in a sequence specific manner.

In another aspect, there is provided a peptide derived from a T cell receptor (TCR) alpha chain transmembrane domain, the peptide comprising at least two basic amino acid residues, wherein all amino acid residues of said peptide are of the “D” isomer configuration.

In another aspect, the invention provides a peptide having an amino acid sequence as set forth in any one of SEQ ID NOS:3 (GLRILLLKV, denoted all-D CP; D-amino acid residues are bold and underlined) and 11 (GFRILLLKV, denoted human all-D CP; D-amino acid residues are bold and underlined). In various embodiments, analogs, fragments and derivatives thereof are provided, wherein all the amino acid residues in said analogs, fragments and derivatives are of the D-isomer configuration. In another particular embodiment, said peptide is amidated at its C-terminus.

In other embodiments, the invention provides lipophilic conjugates comprising a peptide having an amino acid sequence as set forth in any one of SEQ ID NOS:3 and 11 coupled to a fatty acid.

Pharmaceutical Compositions

According to another aspect, the present invention provides a pharmaceutical composition comprising a therapeutically effective amount of a peptide or conjugate according to the principles of the present invention and a pharmaceutically acceptable carrier.

A pharmaceutical composition useful in the practice of the present invention typically contains a peptide or conjugate of the invention formulated into the pharmaceutical composition as a pharmaceutically acceptable salt form. Pharmaceutically acceptable salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Pharmaceutically acceptable salts may be prepared from pharmaceutically acceptable non-toxic bases including inorganic or organic bases. Salts derived from inorganic bases include aluminum, ammonium, calcium, copper, ferric, ferrous, lithium, magnesium, manganic, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

A therapeutically effective amount of a peptide of the invention is an amount that when administered to a patient is capable of inhibiting T cell activation, as specified hereinbelow.

The preparation of pharmaceutical compositions, which contain peptides as active ingredients, is well known in the art. Typically, such compositions are prepared as injectable, either as liquid solutions or suspensions. However, solid forms, which can be suspended or solubilized prior to injection, can also be prepared. The preparation can also be emulsified. The active therapeutic ingredient is mixed with inorganic and/or organic carriers, which are pharmaceutically acceptable and compatible with the active ingredient. Carriers are pharmaceutically acceptable excipients (vehicles) comprising more or less inert substances that are added to a pharmaceutical composition to confer suitable consistency or form to the composition. Suitable carriers are, for example, water, saline, dextrose, glycerol, ethanol, or the like and combinations thereof. In addition, if desired, the composition can contain minor amounts of auxiliary substances such as wetting or emulsifying agents, pH buffering agents, stabilizers, and anti-oxidants, which enhance the effectiveness of the active ingredient. Techniques for formulation and administration of drugs may be found in the latest edition of “Remington's Pharmaceutical Sciences”, Mack Publishing Co., Easton, Pa., which is herein fully incorporated by reference.

For injection, the active ingredients of the pharmaceutical composition may be formulated in aqueous solutions, preferably in physiologically compatible buffers such as Hank's solution, Ringer's solution, or physiological salt buffer.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions. Alternatively, the active ingredient may be in powder form for constitution with a suitable vehicle, e.g., a sterile, pyrogen-free, water-based solution, before use.

The pharmaceutical compositions of the invention are also useful for topical and intralesional application. As used herein, the term “topical” means pertaining to a particular surface area and the topical agent applied to a certain area of said surface will affect only the area to which it is applied. The present invention provides, in some embodiments, topical compositions comprising peptides or conjugates of the invention as active ingredients. In some embodiments, the invention provides compositions consisting essentially of said peptides and conjugates of the invention.

Topical pharmaceutical compositions may comprise, without limitation, non-washable (water-in-oil) creams or washable (oil-in-water) creams, ointments, lotions, gels, suspensions, aqueous or cosolvent solutions, salves, emulsions, wound dressings, coated bandages or other polymer coverings, sprays, aerosols, liposomes and any other pharmaceutically acceptable carrier suitable for administration of the drug topically.

As is well known in the art, the physico-chemical characteristics of the carrier may be manipulated by addition a variety of excipients, including but not limited to thickeners, gelling agents, wetting agents, flocculating agents, suspending agents and the like. These optional excipients will determine the physical characteristics of the resultant formulations such that the application may be more pleasant or convenient. It will be recognized by the skilled artisan that the excipients selected, should preferably enhance and in any case must not interfere with the storage stability of the formulations.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. For example, a cream formulation may comprise in addition to the active compound: (a) a hydrophobic component; (b) a hydrophilic aqueous component; and (c) at least one emulsifying agent. The hydrophobic component of the cream is exemplified by the group consisting of mineral oil, yellow soft paraffin (Vaseline), white soft paraffin (Vaseline), paraffin (hard paraffin), paraffin oil heavy, hydrous wool fat (hydrous lanolin), wool fat (lanolin), wool alcohol (lanolin alcohol), petrolatum and lanolin alcohols, beeswax, cetyl alcohol, almond oil, arachis oil, castor oil, hydrogenated castor oil wax, cottonseed oil, ethyl oleate, olive oil, sesame oil, and mixtures thereof. The hydrophilic aqueous component of the cream is exemplified by water alone, propylene glycol or alternatively any pharmaceutically acceptable buffer or solution. Emulsifying agents are added to the cream in order to stabilize the cream and to prevent the coalescence of the droplets. The emulsifying agent reduces the surface tension and forms a stable, coherent interfacial film, A suitable emulsifying agent may be exemplified by but not limited to the group consisting of cholesterol, cetostearyl alcohol, wool fat (lanolin), wool alcohol (lanolin alcohol), hydrous wool fat (hydrous lanolin), and mixtures thereof.

A topical suspension, for example, may comprise in addition to the active compound: (a) an aqueous medium; and (b) suspending agents or thickeners. Optionally additional excipients are added. Suitable suspending agent or thickeners may be exemplified by but not limited to the group consisting of cellulose derivatives like methylcellulose, hydroxyethylcellulose and hydroxypropyl cellulose, alginic acid and its derivatives, xanthan gum, guar gum, gum arabic, tragacanth, gelatin, acacia, bentonite, starch, microcrystalline cellulose, povidone and mixture thereof. The aqueous suspensions may optionally contain additional excipients e.g. wetting agents, flocculating agents, thickeners, and the like. Suitable wetting agents are exemplified by but not limited to the group consisting of glycerol polyethylene glycol, polypropylene glycol and mixtures thereof, and surfactants. The concentration of the wetting agents in the suspension should be selected to achieve optimum dispersion of the pharmaceutical powders within the suspension with the lowest feasible concentration of the wetting agent. Suitable flocculating agents are exemplified by but not limited to the group consisting of electrolytes, surfactants, and polymers. The suspending agents, wetting agents and flocculating agents are provided in amounts that are effective to form a stable suspension of the pharmaceutically effective agent.

Topical gel formulation, for example, may comprise in addition to the active compound, at least one gelling agent and an acid compound. Suitable gelling agents may be exemplified by but not limited to the group consisting of hydrophilic polymers, natural and synthetic gums, crosslinked proteins and mixture thereof. The polymers may comprise for example hydroxyethylceuulose, hydroxyethyl methylcellulose, methyl cellulose, hydroxypropylcellulose, hydroxypropyl methylcellulose, carboxymethyl cellulose, and similar derivatives of amylose, dextran, chitosan, pullulan, and other polysaccharides; Crosslinked proteins such as albumin, gelatin and collagen; acrylic based polymer gels such as Carbopol, Eudragit and hydroxyethyl methacrylate based gel polymers, polyurethane based gels and mixtures thereof.

Topical pharmaceutical compositions of the present invention may additionally be formulated as a solution. Such a solution comprises, in addition to the active compound, at least one co-solvent exemplified but not limited to the group consisting of water, buffered solutions, organic solvents such as ethyl alcohol, isopropyl alcohol, propylene glycol, polyethylene glycol, glycerin, glycoforol, Cremophor, ethyl lactate, methyl lactate, N-methylpyrrolidone, ethoxylated tocopherol and mixtures thereof.

The composition of the invention may be used for transmucosal, e.g. transdermal delivery. The term “transdennal” delivery as used herein refers to the site of delivery of a pharmaceutical agent. Typically, the delivery is intended to the blood circulation. However, the delivery can include intra-epidermal or intradermal delivery, i.e., to the epidermis or to the dermal layers, respectively, beneath the stratum corneum. For transinucosal administration, penetrants appropriate to the barrier to be permeated are used in the formulation. Such penetrants are generally known in the art.

There are two prevalent types of transdermial patch designs, namely the reservoir type where the drug is contained within a reservoir having a basal surface that is permeable to the drug, and a matrix type, where the drug is dispersed in a polymer layer affixed to the skin. Both types of designs also typically include a backing layer and an inner release liner layer that is removed prior to use. Preparation of such transderrnal patches is within the ability of those of skill in the art; see, for example, U.S. Pat. Nos. 5,560,922, 4,559,222, 5,230,898 and 4,668,232 for examples of patches suitable for transdermal delivery of a therapeutic agent.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

Therapeutic Use

In other aspects, the invention provides methods of treating or preventing the symptoms of a T-cell mediated pathology in a subject in need thereof, comprising administering to the subject a therapeutically effective amount of a peptide or conjugate of the invention.

In one aspect, the present invention provides a method for treating a T cell mediated pathology in a subject in need thereof, comprising administering to the subject a therapeutically effective amount of a diastereomeric peptide derived from a T cell receptor (TCR) alpha chain transmembrane domain, the peptide comprising at least two basic amino acid residues. In one embodiment, the peptide comprises an amino acid sequence as set forth in SEQ ID NO:1, wherein at least one amino acid residue of the diastereomeric peptide is of the D-isomer configuration. In another embodiment, at least two amino acid residues of the diastereomeric peptide are of the D-isomer configuration.

In another embodiment, the diastereomeric peptide has an amino acid sequence as set forth in SEQ ID NO:2. In another embodiment, the peptide comprises an amino acid sequence as set forth in any one of SEQ ID NO:2 and derivatives, fragments, analogs, extensions, conjugates and salts thereof, with the proviso that the peptide or derivative is not a known protein or peptide. In another embodiment, the diastereomeric peptide has an amino acid sequence as set forth in SEQ ID NO:10.

According to certain other particular embodiments, the diastereomeric peptide has an amino acid sequence as set forth in any one of SEQ IUD NOS:12-17, 19-28 and 37-47.

In another embodiment, the diastereomeric peptide is conjugated to a lipophilic moiety.

According to one embodiment of the invention, the lipophilic moiety is a fatty acid selected from the group consisting of saturated, unsaturated, monounsaturated, polyunsaturated and branched fatty acids. According to currently preferred embodiments, the fatty acids consist of at least three, preferably at least six, and more preferably at least eight carbon atoms. In another embodiment, the fatty acid is selected from the group consisting of: octanoic acid (OA), decanoic acid (DA), undecanoic acid (UA), dodecanoic acid (DDA; lauric acid), myristic acid (MA), palmitic acid (PA), stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

In one particular embodiment, the diastereomeric peptide has an amino acid sequence as set forth in SEQ ID NO:29. In other particular embodiments, the diastereomeric peptide has an amino acid sequence as set forth in any one of SEQ ID NOS:30-36 and 48-50.

The term “T-cell mediated pathology” refers to any condition in which an inappropriate or detrimental T cell response is a component of the etiology or pathology of a disease or disorder. The term is intended to include both diseases directly mediated by T cells, and also diseases in which an inappropriate or detrimental T cell response contributes to the production of abnormal antibodies (e.g. autoimmune or allergic diseases associated with production of pathological IgG, IgA or IgE antibodies), as well as graft rejection.

The term “treating” as used herein includes prophylactic and therapeutic uses, and refers to the alleviation of symptoms of a particular disorder in a patient, the improvement of an ascertainable measurement associated with a particular disorder, or the prevention of a particular immune response (such as transplant rejection).

In various embodiments, the subject may be selected from humans and non-human animals.

In one embodiment of the invention, the T cell mediated pathology is a T cell-mediated autoimmune disease, including but not limited to: multiple sclerosis, autoimmune neuritis, systemic lupus erythematosus (SLE), psoriasis, Type I diabetes (IDDM), Sjogren's disease, thyroid disease, myasthenia gravis, sarcoidosis, autoimmune uveitis, inflammatory bowel disease (Crohn's and ulcerative colitis), autoimmune hepatitis and rheumatoid arthritis. In one particular embodiment, the autoimmune disease is rheumatoid arthritis. Other diseases include, but are not limited to, idiopathic thrombocytopenia, scleroderma, alopecia areata, hemolytic anemia, immune-mediated renal disease (e.g. glomerulonephritis), dermatitis and pemphigus.

In another embodiment, the T cell mediated pathology is a T cell-mediated inflammatory disease, including but not limited to inflammatory or allergic diseases such as asthma (particularly allergic asthma), hypersensitivity lung diseases, hypersensitivity pneumonitis, delayed-type hypersensitivity, interstitial lung disease (ILD) (e.g., idiopathic pulmonary fibrosis, or ILD associated with rheumatoid arthritis or other inflammatory diseases).

In other embodiments, the T cell mediated pathology is graft rejection, including allograft rejection and graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of diastereomeric peptides of the invention, that inhibits an immune response, particularly T-cell activation, may be an effective therapy in preventing organ rejection or GVHD. In one embodiment, the immune cells to be transplanted are incubated with a diastereomeric peptide of the invention prior to transplantation.

In another aspect, the invention provides a method of inhibiting T-cell activation in a subject in need thereof, comprising administering to the subject a therapeutically effective amount of a diastereomeric peptide derived from a T cell receptor (TCR) alpha chain transmembrane domain, the peptide comprising at least two basic amino acid residues. In one embodiment, the peptide comprises an amino acid sequence as set forth in SEQ ID NO:1, wherein at least one amino acid residue of the diastereomeric peptide is of the D-isomer configuration. In another embodiment, at least two amino acid residues of the diastereomeric peptide are of the D-isomer configuration.

In another embodiment, the diastereomeric peptide has an amino acid sequence as set forth in SEQ ID NO:2. In another embodiment, the peptide comprises an amino acid sequence as set forth in any one of SEQ ID NO:2 and derivatives, fragments, analogs, extensions, conjugates and salts thereof, with the proviso that the peptide or derivative is not a known protein or peptide. In another embodiment, the diastereomeric peptide has an amino acid sequence as set forth in SEQ ID NO:10.

According to certain other particular embodiments, the diastereomeric peptide has an amino acid sequence as set forth in any one of SEQ ID NOS:12-17, 19-28 and 37-47.

In another embodiment, the diastereomeric peptide is conjugated to a lipophilic moiety.

According to one embodiment of the invention, the lipophilic moiety is a fatty acid selected from the group consisting of saturated, unsaturated, monounsaturated, polyunsaturated and branched fatty acids. According to currently preferred embodiments, the fatty acids consist of at least three, preferably at least six, and more preferably at least eight carbon atoms. In another embodiment, the fatty acid is selected from the group consisting of: octanoic acid (OA), decanoic acid (DA), undecanoic acid (UA), dodecanoic acid (DDA; lauric acid), myristic acid (MA), palmitic acid (PA), stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

In one particular embodiment, the diastereomeric peptide has an amino acid sequence as set forth in SEQ ID NO:29. In other particular embodiments, the diastereomeric peptide has an amino acid sequence as set forth in any one of SEQ ID NOS:30-36 and 48-50.

In another aspect, the invention provides a method of treating a T cell mediated pathology or inhibiting T-cell activation in a subject in need thereof, comprising administering to the subject a therapeutically effective amount of a peptide derived from a T cell receptor (TCR) alpha chain transmembrane domain, the peptide comprising at least two basic amino acid residues, wherein all amino acid residues are of the “D” isomer configuration.

In another aspect, the invention provides a method of treating a T cell mediated pathology or inhibiting T-cell activation in a subject in need thereof, comprising administering to the subject a therapeutically effective amount of a peptide having an amino acid sequence as set forth in any one of SEQ ID NOS:3 and 11, or a lipophilic conjugate thereof.

The pharmaceutical composition can be delivered by a variety of local and systemic delivery routes, including, but not limited to intravenous, intramuscularly, infusion, oral, intranasal, intraperitoneal, subcutaneous, rectal, topical, or into other regions, such as into synovial fluids. Delivery of the composition transdermally is also contemplated, such as by diffusion via a transdermal patch. In one particular embodiment, topical and transdermal administration routes are contemplated, e.g. for DTH and contact dermatitis.

The composition is administered in a manner compatible with the dosage formulation, and in a therapeutically effective amount. The quantity to be administered depends on the subject to be treated, and the capacity of the subject's blood hemostatic system to utilize the active ingredient. Precise amounts of active ingredient required to be administered depend on the judgment of the practitioner and are peculiar to each individual.

The term “therapeutically effective amount” as used herein refers to an amount of the pharmaceutical composition that when administered to a subject is capable of inhibiting T cell activation. Assays for detecting the activity of the peptides of the invention may include, but are not limited to, inhibition of T cell antigen-specific proliferation and inhibition of in vivo disease models including, but not limited to adjuvant arthritis and DTH, as described in the Examples. However, other methods for detecting the inhibition of antigen-specific T cell activation are well known in the art, and may be used for assessing the activity of the peptides of the invention. Preferably, a therapeutically effective amount of a peptide of the present invention is an amount that reduces (inhibits) T cell activation by at least 10 percent, more preferably by at least 50 percent, and most preferably by at least 90 percent, when measured in an in vitro assay or in an in vivo assay. Preferably, a pharmaceutical composition is useful for inhibiting a T cell mediated pathology in a patient as described further herein. In this embodiment, a therapeutically effective amount is an amount that when administered to a patient is sufficient to inhibit, preferably to eradicate, a T cell mediated pathology. A preferred single dose of a peptide derivative or conjugate of the invention is from about 0.8 μg to about 8 mg per kg of body weight, preferably from about 8 μg to about 800 μg per kg of body weight, and more preferably from about 20 μg to about 300 μg per kg of body weight. For example, for topical administration typically a dose equivalent to 5-10 fold of the systemic dose may be used. Typically, the physician will determine the actual dosage which will be most suitable for an individual patient and it will vary with the age, weight and response of the particular patient. There can, of course, be individual instances where higher or lower dosage ranges are merited, and such are within the scope of this invention. The peptide derivative or conjugate of the invention may be administered, for example, as daily or weekly administrations of single doses as described above.

Methods of treating a disease according to the invention may include administration of the pharmaceutical compositions of the present invention as a single active agent, or in combination with additional methods of treatment. The methods of treatment of the invention may be in parallel to, prior to, or following additional methods of treatment.

The following examples are presented in order to more fully illustrate some embodiments of the invention. They should, in no way be construed, however, as limiting the broad scope of the invention.

### EXAMPLES

### A. Diastereomeric Peptides

### Peptide Synthesis and Fluorescent Labeling

Peptides were synthesized by solid phase on PAM-amino acid resin (0.15 meq), as previously described (Kliger et al., 1997; Merrifield et al., 1982). The synthetic peptides were purified (>98% homogeneity) by RP-HPLC on a C₄ column using a linear gradient of 20-60% acetonitrile in 0.05% TFA for 60 min. The peptides were subjected to amino acid analysis and mass spectrometry to confirm their composition. Unless stated otherwise, stock solutions of concentrated peptides in DMSO were used to avoid aggregation of the peptides before use. Resin-bound peptides were treated with 5-carboxytetramethylrhodamine, succinimidyl ester (Rhodamine-SE). The reaction with rhodamine was in DMF containing 2% diisopropylethylamine. The fluorescent probe was used in an excess of 2 equivalents, leading to the formation of resin bound N-terminal rhodamine-labeled peptides (Gerber and Shai, 2000; Gerber et al., 2004). After 1 hr, the resins were washed thoroughly with DMF and then with methylene chloride. All purified peptides were shown to be homogeneous (>98%) by analytical RP-HPLC (Gerber and Shai, 2000).

### CD Spectroscopy

The CD spectra of the peptides were measured in an Aviv 202 spectropolarimeter (Aviv, Lakewood, N.J.). The spectra were scanned with a thermo stated quartz optical cell with a path length of 1 mm. Each spectrum was recorded in 1 nm intervals with an averaging time of 20 sec, at a wavelength range of 260 to 190 nm. The peptides were tested at a 50 μM concentration in either H₂O or 1% LPC micelles.

### Molecular Dynamics Simulation

We used the Insight II software (Accelrys, San Diego, Calif., USA) to model the structure of the all-L CP (wild-type) and the 2D-CP (diastereomer) peptides, based on the structure of an ideal α-helix. In the 2D-CP the Arg and Lys were replaced with their D-enantiomers. These structures were used in a molecular dynamics simulation to compare their structural stability. The simulation was performed on right-handed α-helices for 10 ps using a consistent valence force field (cvff) at 300 K in vacuum. No constraints were used. The structures generated by the simulation were compared to the initial structure after minimization and the RMSD of Carbon Alpha (CA) was calculated using the Decipher module of the Insight II software.

In the lipid bilayer model, the simulation system consists of 107 DMPC molecules, 3655 water molecules and the investigated peptides. In order to keep the simulation box neutral, two water molecules were replaced with two counter C1 anions. The starting conformation of the DMPC membrane was downloaded from the group site of D. P. Tieleman at the University of Calgary (http://moose.bio.ucalgary.ca/index.php?page=Downloads). Simulation and data analysis were carried out using Gromacs 3.2.1 suite (Berendsen et al., 1995). The system was kept under constant pressure and temperature (NPT) weak coupling (Berendsen et al., 1984). Constant pressure of 1 bar was coupled independently, with coupling constant of ιₚ=1.0 ps, to the three direction of the simulation box in order to allow the optimal molecular density. A temperature bath was coupled separately to the ions, water, lipid and protein molecules at 310 k and a coupling constant of τ_{T}=0.1 ps. A cutoff distance for the electrostatic and Lennard-Jones interactions was set to 15 Å and 12 Å, respectively. Atom neighbors list was updated every 20 femtosecond (fs). The long range electrostatic interactions were calculated using the Particle Mesh Ewald (PME) summation (Darden et al., 1993). Bond lengths were constrained using the LINCS algorithm (Hess et al., 1997). A modified version of the united-atoms GROMOS-96 force field was used (van Gunsteren et al., 1996) that was refined for better lipid parameters. All polar hydrogen atoms were treated explicitly, while aliphatic hydrogen atoms were implicitly described in the force field. The flexible simple point charge (SPC) water potential description (Berendsen et al., 1981) was used. The total simulation time was 10 ns, apart from the first 100 ps of restrained run. During the simulation a 2 fs time step was used. The CPU time was approximately 36 hrs per nanosecond on an Intel 2.7 GHz Xeon processor.

### Animals

Two months old female Lewis rats were used. The rats were raised and maintained under pathogen-free conditions in the Animal Breeding Center of the Weizmann Institute of Science. Eight week old female BALB/c mice were maintained under similar pathogen-free conditions. The experiments were performed under the supervision and guidelines of the Institutional Animal Care and Use Committee of The Weizmann Institute of Science.

### Fluorescence Microscopy

Activated A2b T cells (van Eden et al., 1988) (10⁵ cells/sample) were incubated in 100 μl of PBS containing Para-formaldehyde 4% for 15 min on ice. The samples were then washed with cold PBS and centrifuged for 7 min at 1100 rpm. PBS containing BSA 1% was then added at room temperature to prevent non-specific binding. After 30 min FITC-labeled antibody against TCR was added at dilution of 1:100 and incubated for 2.5 hr. For co-localization of TCR with the peptide, Rhodamine 2D-CP was added at a final concentration of 1 μM (stock in DMSO) and incubated for 5 min. Sample were then washed once with PBS and loaded on a microscope slide.

The cells were then observed under a fluorescent confocal microscope. FITC excitation was set at 488 nm, with the laser set at 20% power to minimize bleaching of the fluorophore. Fluorescence data was collected from 505-525 nm. Rhodamine excitation was set at 543 nm, with the laser set at 5% power. Fluorescence data was collected from 560 nm and up.

Fluorescence energy transfer between the FITC (donor) and rhodamine (acceptor) was detected as an increase in FITC fluorescence in an area where the rhodamine probe was bleached. Bleaching was achieved by point excitation at 543 nm for 6 sec with the laser set to 100%. To verify that the increase in FITC fluorescence is not due to auto fluorescence, bleaching was performed using the 488 nm laser first and only then at 543 nm. No signal was observed in either 505-525 nm or 560<, eliminating the possibility of auto fluorescence.

Co-Immunoprecipitation of Fluorescence-Labeled Peptides with TCR Molecules.

Activated A2b T cells (2×10⁶) were cultured for 1 hr at 37° C. in the presence of CP or 2D-CP (25 μg/ml), or 2G-CP, and lysed for 15 min on ice in 0.1 ml lysis buffer (Adachi et al., 1996). Insoluble material was removed by centrifugation at 10.000 g for 10 min at 4° C. The lysate was then incubated overnight with Protein A-plus Agarose beads (Santa Cruz Biotechnology Inc., Santa Cruz, Calif., USA) bound to antibodies to the rat TCR or MHC class 1. The antibodies reactive against the rat TCR (clone R73) or HSP60 (clone LKI) were purified from the respective hybridomas at our lab; antibodies to rat CD28, actin and the rat MHC class 1 were purchased from Serotec (Oxford, UK). After an overnight incubation at 4° C., the beads were washed with lysis buffer, boiled for 10 minutes and the protein supernatant run in a 4-20% SDS-PAGE. The presence of co-immunoprecipitated peptide was detected by the Typhoon 9400 variable mode imager.

### T-Cell Proliferation

T cell proliferation assays were performed using LNC from rats immunized with Mt. Popliteal and inguinal LNC were removed 26 days after the injection of Mt in incomplete Freund's adjuvant (IFA), when strong T-cell responses to PPD and the Mt176-90 peptide of the 65 kDa heat shock protein (HSP65) (Quintana et al., 2002) are detectable. LNC were cultured at a concentration of 2×10⁵ cells per well; 5×10⁴ A2b T cells were stimulated in the presence of irradiated 5×10⁵ thymic antigen presenting cells per well, prepared as previously described (van Eden et al., 1985). The cells were plated in quadruplicates in 200 μl round bottom microtiter wells, with or without antigen, in the presence of various concentrations of the peptides under study and PPD or Mt176-90. Cultures were incubated for 72 hr at 37° C. in a humidified atmosphere of 7.5% CO₂. T-cell responses were detected by the incorporation of [methyl-³H]-thymidine (Amersham, Buckinghamshire, UK; 1 μCi/well), added during the last 18 hr of incubation. The results of T-cell proliferation experiments are shown as the % of inhibition relative to the T cell proliferation triggered with antigen stimulation in the absence of peptides.

### Induction and Assessment of AA

To test the effect of CP on T-cell activation in vivo, the experimental T-cell mediated autoimmune disease adjuvant arthritis (AA) was used. AA was induced by injecting 50 μl of Mt suspended in IFA (10 mg/ml) at the base of the tail. At the time of AA induction, each rat also received 100 μg of all-L CP, 2D-CP or 2G CP control peptide (or PBS) dissolved in 50 μl of IFA and mixed with Mt/IFA used to induce AA. The day of AA induction was designated as day 0. Disease severity was assessed by direct observation of all 4 limbs in each animal. A relative score between 0 and 4 was assigned to each limb, based on the degree of joint inflammation, redness and deformity; thus the maximum possible score for an individual animal was 16 (Quintana et al., 2002). The mean AA score (±SEM) is shown for each experimental group. Arthritis was also quantified by measuring hind limb diameter with a caliper. Measurements were taken on the day of the induction of AA and 26 days later (at the peak of AA); the results are presented as the mean±SEM of the difference between the two values for all the animals in each group. The person who scored the disease was blinded to the identity of the groups.

### Delayed Type Hypersensitivity to PPD

Twenty μl of PPD (0.5 mg/ml in PBS) were injected intradermally into the pinna of the right ear of each rat on day 16 after AA induction; 20 μl of sterile PBS were injected in the left ear as control. The thickness of the ear was measured 48 hr later using a vernier caliper and expressed as the difference between the right and the left ear (Quintana et al., 2005).

### Delayed Type Hypersensitivity to Oxazalone

Groups of 5 female inbred BALB/c mice (The Jackson Laboratory) were sensitized on the shaved abdominal skin with 100 μl of 2% Oxazalone dissolved in acetone/olive oil [4:1 (vol/vol)] applied topically. DTH sensitivity was elicited 5 days later by challenging the mice with 20 μl of 0.5% Oxazalone in acetone/olive oil, 10 μl administered topically to each side of the ear. A constant area of the ear was measured immediately before challenge and 24 h after challenge with a Mitutoyo engineer's micrometer. The individual measuring ear swelling was unaware of the identity of the groups of mice. The DTH reaction is presented as the increment of ear swelling after challenge expressed as the mean±SEM in units of 10-2 mm. One hour after the challenge the mice's ears were treated topically with either all-L CP or with 2D-CP, both dissolved in 40 μl DMSO. The activity of the peptide treatments was compared to treatment with DMSO alone for “untreated mice” and Dexamethasone (100 μg/ml in saline) as a positive control.

### Statistical Significance

The InStat 2.01 program (Graph Pad Software, San Diego, Calif.) was used for statistical analysis. The Student t test and the Mann-Whitney U test (two tailed) were conducted to assay significant differences between the different experimental groups.

### Example 1

### 2D-CP Lacks a Stable α-Helical Structure

Recently, the present inventors have shown that the inhibitory activity of CP on T cell activation is independent of peptide chirality. To analyze the differential contribution of secondary structure and side-chain sequence to its ability to interact with the TCR/CD3 complex and interfere with T cell activation, the inventors have replaced the two positive residues of CP (Arg and Lys) with their D-enantiomers (2D-CP). The insertion of D aa in an L peptide has been described to destabilize the secondary structure while keeping the aa sequence (FIG. 1). To test for sequence specificity, the inventors synthesized a known mutant in which the two positive residues were mutated to glycine (Gerber et al., 2005; Manolios et al., 1997). The designation and aa sequence of the CP peptides used in Examples 1-5 are as follows:

All-L CP (wild type): GLRILLLKV (SEQ ID NO: 1).
2D-CP (diastereomer): GLRILLLKV (SEQ ID NO:2; D-aa are bold and underlined).
2G-CP (control): GLGILLLGV (SEQ ID NO:51; the Gly mutations are in bold italic font).

The introduction of two D aa into the CP peptide is expected to destabilize the secondary structure. The inventors studied the circular dichroism (CD) spectrum of the 2D-CP peptide in two experimental conditions: in H₂O and in 1% lysophosphatidyl-choline (LPC) micelles (FIG. 1B). The latter mimics the hydrophobic environment of the membrane. The all-L CP had α-helical secondary structure in micelles, similar to previous reports (Gerber et al., 2005; Ali et al., 2001). No significant secondary structure for the 2D-CP peptide was found, as expected (FIG. 1B).

The inventors further compared the stability of the secondary structures of all-L CP and 2D-CP by running a molecular dynamics simulation, as detailed below.

(i) Molecular dynamics simulation in vacuum: the comparison of the secondary structures resulting from the molecular dynamics simulation indicates that all-L CP maintains its α-helical structure, which is lost in the 2D-CP mutant (FIG. 1C). The inventors used the Decipher analysis package in the Insight II software to compare the stability of the all-L CP and 2D-CP peptides. The RMSD increases as a molecular structure departs from its initial structure, and it reaches a plateau once a new stable conformation has been acquired. FIG. 1C shows both the all-L CP and 2D-CP after they reached equilibrium. The all-L CP maintained its helical structure while the 2D-CP lost its helicity. When the RMSD of CA for the two peptides were compared, it was found that both the wild-type all-L CP and 2D-CP equilibrated after about 4 ps in vacuum. The RMSD calculated over the equilibrated phase is 2.16 and 3.39 for all-L CP and 2D-CP, respectively. The RMSD of 2D-CP is 60% higher than that of all-L CP, again indicating that 2D-CP displays a secondary structure that is less stable than that of all-L CP.

(ii) Molecular dynamics simulation in a lipid bilayer model: the inventors further compared the stability of the secondary structures of all-L CP and 2D-CP by running a molecular dynamics simulation in a lipid bilayer model for 10 nano-seconds. The comparison of the secondary structures resulting from the molecular dynamics simulation indicates that all-L CP maintains its α-helical structure, which is lost in the 2D-CP mutant. The stability of the all-L CP and 2D-CP peptides was compared at equilibrium. The RMSD increases as a molecular structure departs from its initial structure, and it reaches a plateau once a new stable conformation has been acquired. The all-L CP maintained its helical structure while the 2D-CP lost its helicity. When the RMSD of CA was compared for the two peptides, it was found that both the wild-type all-L CP and 2D-CP equilibrated after about 4 ns in the lipid bilayer. The RMSD calculated over the equilibrated phase is 0.25 nm (FIG. 1D) and 0.4 nm (FIG. 1E) for all-L CP and 2D-CP, respectively. The RMSD of 2D-CP is 62.5% higher than that of all-L CP, again indicating that 2D-CP displays a secondary structure that is less stable than that of all-L CP. There was no significant difference in the effect of the two peptides on the membrane density after insertion into the bilayer.

In conclusion, the results of this computational analysis together with those of the CD spectra indicate that the two D aa disrupt the right-handed α-helical structure adopted by the all-L CP peptide.

### Example 2

### The α-Helical Structure of CP is Not Required for T Cell Binding and Localization

The CP peptide has been described to insert itself into the CD3/TCR complex and interfere with the activation of T cells triggered by their cognate antigen (Manolios et al., 1997; Wang et al., 2002; Wang et al., 2002b). The contribution of the secondary structure to the CP-CD3/TCR interactions was analyzed by studying the localization of rhodamine-labeled 2D-CP and TCR-specific FITC-labeled antibodies (αTCR-FITC) on the T-cell membrane. FIG. 2 depicts the co-localization of αTCR-FITC and Rhodamine 2D-CP (FIG. 2), suggesting that the 2D-CP analog inserts into the T-cell membrane and co-localizes with the TCR, as was seen with wild-type CP (Gerber et al., 2005; Wang et al., 2002b).

To confirm these co-localization results, the inventors performed fluorescence energy transfer experiments between Rhodamine 2D-CP and αTCR-FITC. Using a 543 nm laser a point on the T-cell membrane that exhibited high intensity of both Rhodamine 2D-CP and αTCR-FITC was irradiated, bleaching the signal produced by the Rhodamine 2D-CP but leaving intact the emission produced by the αTCR-FITC. This procedure led to a significant increase in the fluorescence of the αTCR-FITC shown in FIG. 2D. To rule out auto-fluorescence as a source of increased signal in the 505-525 mm range, two controls were used. First, the same position was bleached with the 488 nm laser. This resulted in complete bleaching of the signal, suggesting that the fluorescence increase seen above was generated by the FITC probe and did not result from auto-fluorescence in the sample. Second, the inventors chose a point on the surface of cells stained with Rhodamine 2D-CP and αTCR-FITC that showed no fluorescent signal and bleached it with the 543 nm laser. No increase in the FITC fluorescence was observed, ruling out auto-fluorescence as a source of fluorescence in the range of FITC. All in all, these results confirm that wild-type CP and its 2D-CP stereoisomer show the same localization in the T-cell membrane regardless of their differences in secondary structure.

Next, co-immunoprecipitation experiments were performed. The affinity of the TCR/CP, the TCR/2D-CP and TCR/2G-CP interactions were compared by performing a series of co-precipitation experiments using different concentrations of Rho-labeled CP, 2D-CP and 2G-CP peptides. FIG. 6 shows that both CP and 2D-CP can be co-precipitated with the TCR, although the TCR/CP interaction seems to be of higher affinity. Both CP and 2D-CP interact with the TCR significantly more than the 2G-CP mutant. On the other hand, none of the peptides significantly co-precipitated together with the MHC I molecule that was used as a control receptor. These results further support specific interaction of 2D-CP with the TCR molecule.

### Example 3

### 2D-CP Interferes with T-Cell Activation in Vitro

The co-localization studies presented herein (FIG. 2) suggested that the secondary structure of CP was not essential for its ability to insert into the T-cell membrane and interact with the CD3/TCR complex. To test whether the secondary structure of CP contributed to its interference with the activation of T cells by antigen, the inventors isolated lymph node cells (LNC) from Mycobacterium tuberculosis (Mt)-immunized rats and activated the T cells in vitro with tuberculin-purified protein derivative (PPD) or with the Mt176-90 peptide. Both PPD and Mt176-90 have been reported to induce a strong T-cell proliferative response in the LNC of Mt-immunized rats (Quintana et al., 2002; Quintana et al., 2003). Both wild-type all-L CP and 2D-CP inhibited T-cell proliferation to PPD and to Mt176-90 in a dose-dependent manner (FIG. 3). Note, however, that at lower concentrations, the inhibition of 2D-CP was somewhat greater (p<0.02) than that produced by all-L CP (FIG. 3B). Conversely, the 2G CP mutant did not have any inhibitory effect on T-cell proliferation, suggesting that inhibition was sequence specific and that critical molecular interactions were perturbed by the substitution of two positive aa for Gly residues (see FIG. 1). None of the peptides (all-L CP, 2D-CP or 2G CP) had a cytotoxic effect when incubated with the target cells, ruling out that the effects on antigen-triggered proliferation were due to cell death. Thus the secondary structure of CP does not seem to be required for its inhibitory effects on T cell activation, which remained dependent on precise side-chain interactions as evidenced by the 2G CP mutant.

### Example 4

### 2D-CP Inhibits T-Cell Immunity in Vivo

Next, the contribution of the secondary structure and the side-chains to the inhibitory effects of CP on T cell activation In vivo was analyzed, using the experimental autoimmune disease Adjuvant Arthritis (AA). Immunization of Lewis rats with Mt triggers AA, an autoimmune disease driven by Mt-specific T cells that cross-react with self-antigen (Holoshitz et al., 1984; Holoshitz et al., 1983). PPD and Mt176-90 are targeted by the arthritogenic T-cell response (Anderton et al., 1994); indeed immunomodulatory therapies that inhibit the progression of arthritis have been associated with a decreased T cell response to PPD and Mt176-90 and with a diminished delayed type hypersensitivity (DTH) response to PPD (van Eden et al., 1985).

The administration of all-L CP or 2D-CP with Mt at the time of AA induction led to a significantly milder arthritis, both in terms of clinical score (FIG. 4A) and of paw swelling (FIG. 4B); the 2G CP peptide did not have an effect on AA. The mean maximum score was 12±0.3 in the 2G CP-treated rats, compared with 6.6±0.7 in the 2D-CP-treated rats and 7.7±0.7 in the all-L CP group (p<0.05 for the all-L CP and the 2D-CP groups when compared to the 2G CP group). The effect of 2D-CP on AA was dose dependent, since an increase of the dose of 2D-CP to 900 μg per rat led to a further 25% decrease in the AA score (data not shown) and a 50% improvement in paw swelling (FIG. 4C).

The study of the DTH response to PPD 16 days after AA induction revealed that the administration of wild-type all-L CP or 2D-CP led to a 39% and 51% reduction in the DTH response to PPD, respectively; treatment with 2G CP led only to an 8.5% inhibition of the DTH response.

Taken together, these results indicate that both the 2D-CP and wild-type all-L CP can interfere in vivo with T-cell activation, in a sequence specific manner that was, nevertheless, independent of the secondary structure of the peptide.

### Example 5

### 2D-CP Decreases the DTH Response in a Therapeutic Setting

To test whether TCR trans-membrane peptides could inhibit the elicitation of existing DTH reactivity, groups of naïve BALB/c mice were sensitized to 2% oxazalone. Five days later, the mice were challenged with 0.5% oxazalone administered to the ear. An hour after the challenge, the mice were treated with Dimethylsulfoxide (DMSO), all-L CP 150 μg (6 mg/kg) in DMSO, 2D-CP 150 μg (6 mg/kg) in DMSO, or dexamethasone, applied topically. On the following day, ear thickness was measured the swelling of DMSO treated mice (0.33±0.01 mm) was compared to that of mice treated with all-L CP (0.30±0.01 mm), 2D-CP (0.27±0.01 mm) and Dexamethasone (0.15±0.02 mm). A significant reduction in ear swelling was observed in the mice that had been treated with 2D-CP (p<0.1 when compared to the DMSO group); indeed, 2D-CP caused twice the reduction produced by treatment with all-L CP (p<0.05 when compared to the DMSO group) (FIG. 5). Thus, 2D-CP can inhibit a T-cell mediated immune reaction in subjects already sensitized to the antigen.

### Example 6

### CP Lipophilic Conjugates

### (i) Materials

4-Methyl benzhydrylamine resin (BHA) and butyloxycarbonyl (Boc) amino acids are purchased from Calbiochem-Novabiochem Co. (La Jolla, Calif., USA). Other reagents used for peptide synthesis include trifluoroacetic acid (TFA, Sigma), N,N-diisopropylethylamine (DIEA, Sigma), dicyclohexylcarbodiimide (DCC, Fluka), 1-hydroxybenzotriazole (1-HOBT, Pierce), and dimethylformamide (DMF, peptide synthesis grade, Biolab, IL). All other reagents are of analytical grade. Buffers are prepared in double-distilled water.

### (ii) Peptide Synthesis, Acylation and Purification

Peptides are synthesized by a solid phase method on 4-methyl benzhydrylamine resin (BHA) (0.05 meq) (Merrifield et. al., 1982; Shai et. al., 1990). The resin-bound peptides are cleaved from the resin by hydrogen fluoride (HF) and, after HF evaporation, and washing with dry ether, extracted with 50% acetonitrile/water. HF cleavage of the peptides bound to BHA resin result in C-terminus amidated peptides. Crude peptide preparations are subjected to RP-HPLC. The synthesized peptides are further purified by RP-HPLC on a C₁₈ reverse phase Bio-Rad semi-preparative column (250×10 mm, 300 nm pore size, 5-μm particle size). The column is eluted in 40 min, using a linear gradient of 25-60% acetonitrile in water, both containing 0.05% TFA (v/v), at a flow rate of 1.8 ml/min. The purified peptides are then subjected to analytical HPLC, amino acid analysis and electrospray mass spectroscopy to confirm their composition and molecular weight. The fatty acid is conjugated to the N-terminus of the peptides using the same protocol used to attach protected amino acids for peptide synthesis.

The following lipopeptides are synthesized as described herein. Decanoic acid (DA), undecanoic (UA), and octanoic acid (OA) are conjugated to diastereomeric CP-derived peptides to yield the following lipophilic conjugates:

Octyl-GLRILLLKV
Octyl-RILLLK-Octyl
Decyl-RILLLK
Octyl-GLRILLLKV
Decyl-GFRILLLKVAGF
Undecyl-GFRILLLKVAGF
GLKILLLKV-Decyl
Decyl-RILLLK-Decyl

(iii) in vitro assays

The lipopeptides are then examined for their ability to interfere with T-cell activation in vitro as described in Example 3.

(iv) in vivo assays

The lipopeptides are subjected to in vivo assays of T cell activation, as described in Examples 4 and 5.

### B. All-D Peptides

Peptide Synthesis and Fluorescent Labeling Peptides were synthesized by solid phase on PAM-amino acid resin (0.15 meq). The synthetic peptides were purified (>98% homogeneity) by RP-HPLC on a C₄ column using a linear gradient of 20-60% acetonitrile in 0.05% TFA for 60 min. The peptides were subjected to amino acid analysis and mass spectrometry to confirm their composition. Unless stated otherwise, stock solutions of concentrated peptides in DMSO were used to avoid aggregation of the peptides before use. The final concentration of DMSO in each experiment had no effect on the system under investigation. Resin-bound peptides were treated with 4-chloro-7-nitrobenz-2-oxa-1,3-diazole fluoride (NBD-F) or 5-carboxytetramethylrhodamine, succinimidyl ester (5-TAMRA, SE (Rhodamine-SE), respectively. The reaction with NBD-F took place in DMF alone, and the reaction with rhodamine in DMF containing 2% diisopropylethylamine. The fluorescent probes were used in excess of 2 equivalents, leading to the formation of resin bound N-terminal NBD or rhodamine peptides. After 1 h, the resins were washed thoroughly with DMF and then with methylene chloride. All purified peptides were shown to be homogeneous (>98%) by analytical RP-HPLC.

Table 3 shows the sequences and designations of the peptides used in examples 7-10.

TABLE 3
Peptides' designation and sequence.

| | | SEQ ID NO. |
|---|---|---|
| | | (unlabeled |
| | | C′-amidated |
| Peptide Designation | Sequence | peptide) |
| L-CP (amidated) | X-GLRILLLKV-NH₂ | 52 |
| D-CP (amidated) | X-GLRILLLKV-NH₂ | 53 |
| 2G CP (amidated) | X-GLGILLLGV-NH₂ | 54 |

D-amino acids are underlined and mutations are in bold.

X₁=-NH₃, unlabeled peptide.

X2=-NH-Rhodamine, Rhodamine labeled peptide.

X3-NH-NBD, NBD labeled peptide.

The peptides were amidated at their C terminus.

### Circular Dichroism (CD) Spectroscopy

The CD spectra of the peptides were measured in an Aviv 202 spectropolarimeter. The spectra were scanned with a thermo stated quartz optical cell with a path length of 1 mm. Each spectrum was recorded in 1 nm intervals with an averaging time of 20 sec, at a wavelength range of 260 to 190 nm. The peptides were scanned at a 100 μM concentration in 1% LPC micelles.

### Animals

Two-month old female Lewis rats were used. The rats were raised and maintained under pathogen-free conditions in the Animal Breeding Center of the Weizmann Institute of Science. The experiments were performed under the supervision and guidelines of the Animal Welfare Committee,

### T-Cell Proliferation

T cell proliferation assays were performed using either lymph node cells (LNC) or the A2b T cell line, which reacts with the Mt176-90 peptide. Popliteal and inguinal LNC were removed 26 days after the injection of Mycobacterium tuberculosis (Mt) in incomplete Freund's adjuvant (IFA), when strong T cell responses to PPD and Mt176-90 are detectable. LNC were cultured at a concentration of 2×10⁵ cells per well; 5×10⁴ A2b T cells were stimulated in the presence of irradiated 5×10⁵ thymic antigen presenting cells (APC) per well, prepared as previously described. The cells were plated in quadruplicates in 200 μl round bottom microtiter wells, with or without antigen, in the presence of various concentrations of the peptides under study. Cultures were incubated for 72 hr at 37° C. in a humidified atmosphere of 7.5% CO₂. T-cell responses were detected by the incorporation of [methyl-3H]-thymidine (Arnersham, Buckinghamshire, UK; 1 μCi/well), added during the last 18 hr of incubation. The results of T cell proliferation experiments are shown as the % of inhibition of the T cell proliferation triggered by the antigen in the absence of peptides.

### Induction and Assessment of Adjuvant Arthritis (AA)

To test the effect of CP on T-cell activation in vivo, we used AA as a model system. AA was induced by injecting 50 μl of Mt suspended in IFA (0.5 mg/ml) at the base of the tail. At the time of AA induction, each rat also received 100 μg of L-CP, D-CP or 2G CP control peptide (or PBS) dissolved in 50 μl of IFA and mixed with Mt/IFA used to induce AA. The day of AA induction was designated as day 0. Disease severity was assessed by direct observation of all 4 limbs in each animal. A relative score between 0 and 4 was assigned to each limb, based on the degree of joint inflammation, redness and deformity; thus the maximum possible score for an individual animal was 16. The mean AA score (±SEM) is shown for each experimental group. Arthritis was also quantified by measuring hind limb diameter with a caliper. Measurements were taken on the day of the induction of AA and 26 days later (at the peak of AA); the results are presented as the mean±SEM of the difference between the two values for all the animals in each group. The person who scored the disease was blinded to the identity of the groups.

### Delayed Type Hypersensitivity (DTH)

Twenty μl of PPD (0.5 mg/ml in PBS) were injected intradermally into the pinna of the right ear on day 16 after AA induction; 20 μl of sterile PBS were injected in the left ear as control. The thickness of the ear was measured 48 hr later using a vernier caliper and expressed as the difference between the right and the left ear.

### Fluorescence Microscopy

Activated T cells (10⁵ cells/sample) were incubated in 100 μl of PBS containing Para-formaldehyde 4% for 15 min on ice. The samples were then washed with cold PBS and centrifuged for 7 min at 1100 rpm. PBS containing BSA 1% was then added at ambient temperature to prevent non-specific binding. After 30 min FITC-labeled antibody against TCR was added at 1:100 dilution and incubated for 2.5 hrs. For co-localization of TCR with the peptides, L-CP-Rho, D-CP-Rho or 2D CP-Rho were added at a final concentration of 1 μM (stock in DMSO) and incubated for 5 min. Sample were then washed once with PBS and loaded on a microscope slide.

The cells were then observed under a fluorescent confocal microscope. FITC excitation was set at 488 nm, with the laser set at 20% power to minimize bleaching of the fluorophore. Fluorescence data was collected from 505-525 nm. Rhodamine excitation was set at 543 nm, with the laser set at 5% power. Fluorescence data was collected from 560 nm and up.

FRET between the FITC (donor) and rhodamine (acceptor) was observed as increase in FITC fluorescence in an area where the rhodamine probe was bleached. Bleaching was achieved by point excitation at 543 nm for 6 sec with the laser set to 100%. To verify that the increase in FITC fluorescence is not due to auto fluorescence, we bleached using the 488 nm laser and only then at 543 nm. No signal was observed in either 505-525 nm or 560<, eliminating the possibility of auto fluorescence.

### Example 7

### D-CP is a Structural Mirror Image of L-CP

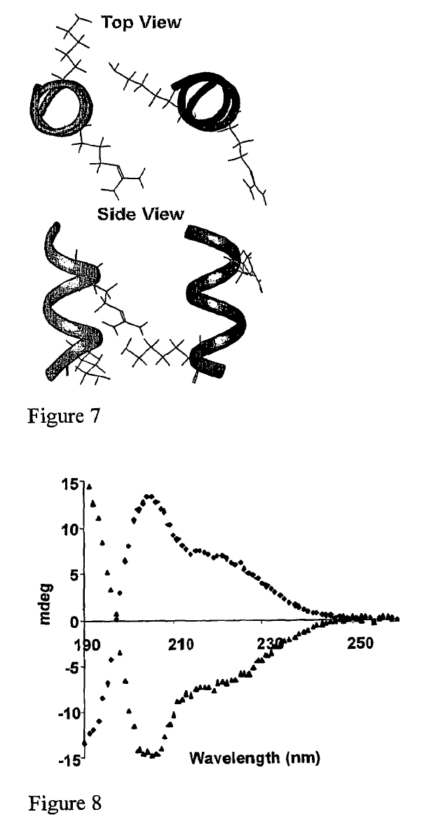

Three CP peptides chemically synthesized: wild type L-CP, which has been shown to inhibit T-cell activation by the target antigen; D-CP, which is a mirror image of the first; and an inactive mutated peptide (2G CP). Table 3 shows the peptide sequences and designations, and FIG. 7 visually demonstrates the structural difference between the two stereoisomers, assuming a canonical helical structure. Note that the two bulky positive side chains on D-CP are facing in the opposite direction of those in L-CP.

Circular dichroism experiments were performed to ensure that the secondary structure of the D-CP was indeed a mirror image of the L-CP. The experiments were performed in a zwitterionic detergent (1% LPC in H₂O) to simulate a membrane environment, as described in Melnyk et al., 2004. The spectrum of the D-CP was found to be exactly a mirror image of the L-CP (FIG. 8); both are partially helical. Note that both peptides are 9 aa long, hence their structure is likely to be less stable than that in the context of the full length protein.

### Example 8

### D-CP Interferes with T-Cell Activation as Does L-CP

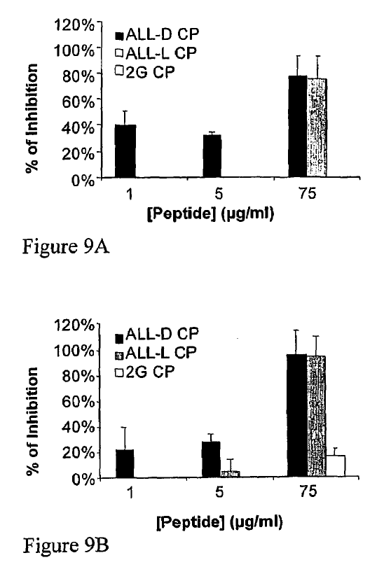

We studied the T-cell response of LNC from Mt-immunized rats to the Mt antigen PPD or to the Mt176-90 peptide; these antigens are known to induce strong proliferative responses from T cells in the draining LNC of AA rats. FIGS. 9A and 9B show that L-CP and D-CP inhibited the T-cell proliferative responses to PPD and to Mt176-90 in a dose-dependent manner. Moreover, there was no inhibitory effect for the 2G CP, suggesting that the inhibition is sequence specific and that critical molecular interactions were perturbed by the substitution of two positive aa for Gly residues (see Table 3). L-CP, D-CP and 2G CP showed no cytotoxicity when incubated with cells, excluding the possibility that inhibitory effects of the L and D-CP peptides on antigen-triggered proliferation were due to cell death. Interestingly, the inhibition of D-CP is consistently higher than that of L-CP at the lower concentrations.

### Example 9

### D-CP Inhibits T-Cell Immunity in Vivo to the Same Extent as L-CP

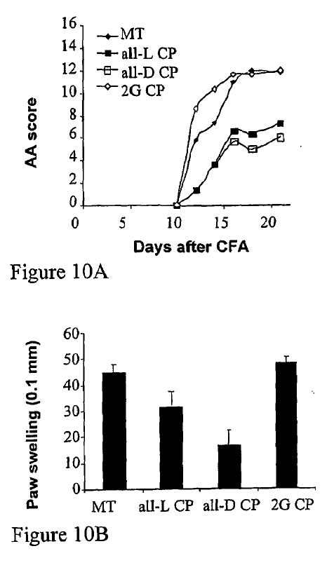

To test the inhibitory effects of CP on the activation of specific T cells in vivo, we used the adjuvant arthritis (AA) model. Immunization of Lewis rats with Mt in oil triggers AA, an experimental autoimmune disease driven by Mt-specific T cells cross-reactive with self-antigens. Mt176-90-specific T cells are detectable upon induction of AA; indeed the A2b T-cell clone cross-reacts with cartilage and mediates AA. Since L and D-CP inhibited the T-cell response of primed LNC and of clone A2b to PPD and Mt17-90 in vitro (FIG. 9), we also investigated the effects of L- or D-CP on the in vivo activation of the T cells that drive AA. D-CP or L-CP administered with the antigen at the time of AA induction led to a significantly milder arthritis, both in terms of clinical score (FIG. 10A) and of ankle swelling (FIG. 10B). The control peptide 2G CP did not inhibit AA. The mean maximum score was 12±0.3 in the control-treated rats, compared with 6±0.7 in the D-CP-treated rats and 7.3±0.7 in the L-CP-treated rats (p<0.05 for the L and D-CP groups compared to the control groups).

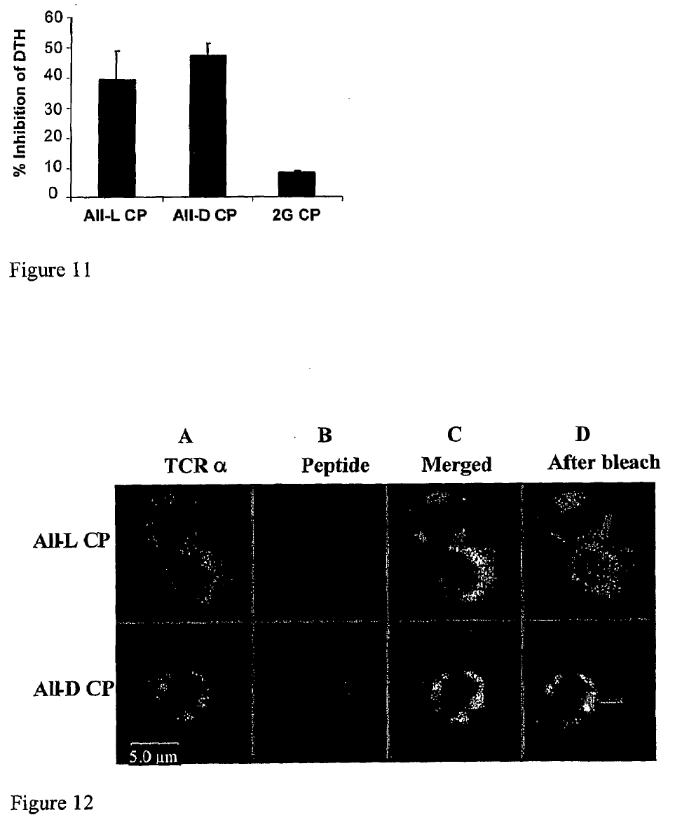

The activity of the T cells that mediate AA can also be detected in vivo by studying the delayed type hypersensitivity (DTH) response to PPD. We studied the DTH response to PPD 16 days after AA induction in rats treated with the three peptides. FIG. 11 shows that the administration of D-CP or L-CP led to a 48% and 39% reduction in the DTH response to PPD, respectively, while the inhibition caused by treatment with the 2G CP peptides was less than 10%.

Taken together, these results indicate that both the D and L-CP can interfere in vivo with T-cell activation induced by specific antigens. This interference led to milder AA (FIGS. 10A and 10B) and decreased DTH reactivity (FIG. 11) in response to Mt antigens. Moreover, it seems that in vivo the effect of D-CP is greater than that of L-CP.

### Example 10

### Co-Localization of L-CP and D-CP with the TCR

The CP peptides function by uncoupling the signal between TCR and CD3, therefore they should co-localize with the receptor complex. To test this hypothesis, we labeled the T cells with FITC-labeled antibodies against TCR and either L-CP or D-CP labeled with rhodamine (FIG. 12). The labeling of the TCR demonstrated the capping phenomenon characteristic of activated T cells. There was almost complete overlap between TCR and either L or D analogues of CP. These results suggest that the CP analogues bind to T cell membrane and co-localize with the TCR within the capping regions. We believe that the labeling does not affect the localization since the same results were obtained with NBD-labeled peptides and PE-labeled TCR.

To corroborate the co-localization results, we performed a series of bleaching experiments that demonstrated fluorescence energy transfer between the CP peptides and the TCR. We bleached a point on the membrane of the cells, which exhibited high intensity of both Rhodamine and FITC, using the 543 nm laser. Thus, the CP rhodamine-labeled peptides were bleached while the TCRα-FITC was unaffected. This procedure resulted in a significant increase in the fluorescence of the TCRα-FITC, pointed out by the arrows in FIG. 12. Thus, we could conclude that fluorescence energy transfer occurs between the TCRα and L-CP or D-CP peptides. To eliminate the possibility of autofluorescence as the source of increased signal in the range of 505 μm to 525 nm, we performed two controls. First, we bleached the same position with the 488 nm laser. This resulted in complete bleach of the signal, suggesting that the fluorescence increase described above is generated by the FITC probe rather than being an artifact of autofluorescence from the sample. Next, we bleached with the 543 mm laser at a position with almost no signal. The cell remained impervious and we observed no increase in the FITC fluorescence, eliminating the possibility of autofluorescence at the same spectral range of FITC.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

### REFERENCES

Adachi et al. (1996). EMBO J. 15: 1534-1541.
Ali M et al., (2001). Letters in Peptide Science 8: 227-233.
Anderton S M et al., (1994). J Immunol 152: 3656-3664.
Bender V et al., (2004). Journal of Biological Chemistry 279: 54002-54007.
Berendsen et al., (1981) Intermolecular Forces, chapter Interaction models for water in relation to protein hydration. Dordrecht: D. Reidel Publishing Company Dordrecht. pp. 331-342.
Berendsen et al., (1984). Journal of Chemical Physics 81: 3684-3690.
Berendsen et al., (1995). Computer Physics Communications 91: 43-56.
Bodanszky, M., Principles of Peptide Synthesis, Springer-Verlag, 1984.
Darden et al., (1993). Journal of Chemical Physics 98: 10089-10092.
Enk, A. H. & Knop, J. (2000). Int. Arch. Allergy Immununol. 123, 275-281.
Gerber D, et al. (2004). J Biol Chem 279: 48224-48230.
Gerber D, Shai Y (2000). J Biol Chem 275: 23602-23607.
Gobel, T. W et al., (1994). Proc. Natl. Acad. Sci. U.S.A 91, 1094-1098.
Hess et al., (1997). Journal of Computational Chemistry 18: 1463-1472.
Holoshitz J et al., (1983). Science 219: 56-58.
Holoshitz J et al., (1984). J Clin Invest 73: 211-215.
Kliger Y et al. (1997). J Biol Chem 272: 13496-13505.
Manolios et al. (1997). Nat Med 3: 84-88.
Manolios, N. et al., (1990). Science 249, 274-277.
Melnyk et al., (2004). J. Biol. Chem. 279, 16591-16597.
Merrifield R B et al., (1982). Biochemistry 21: 5020-5031.
Merrifield, (1963). J. Am. Chem. Soc., 85:2149.
Partula, S. et al., (1996). J. Immol. 157, 207-212.
Quintana F J et al., (2002). J Immunol 169: 3422-3428.
Quintana F J et al., (2003). J Immunol 171: 3533-3541.
Quintana F J et al., (2005). J Clin Invest. 115(8):2149-58.
Remington's Pharmaceutical Sciences, 18th ed., Mack Publishing Company (1990).
van Eden W et al. (1985). Proc Natl Acad Sci USA 82: 5117-5120.
van Eden W et al. (1988). Nature 331: 171-173.
van Gunsteren et al., (1996) Biomolecular Simulation: The GROMOS96 manual and user guide. Z\ddot{}urich, Switzerland: Hochschulverlag AG an der ETH Z\ddot{}urich.
Wang et al., (2002). Cell Immunol 215: 12-19.
Wang et al. (2002b). Clin Immunol 105: 199-207.

1-54. (canceled)

55. A diastereomeric peptide derived from a T cell receptor (TCR) alpha chain transmembrane domain, the peptide comprising at least two basic amino acid residues.

56. The diastereomeric peptide of claim 55, wherein at least two amino acid residues of the diastereomeric peptide are of the D-isomer configuration.

57. The diastereomeric peptide of claim 55, wherein the two basic amino acid residues are separated by 3-5 hydrophobic amino acid residues.

58. The diastereomeric peptide of claim 57, wherein the two basic amino acid residues are separated by four hydrophobic amino acid residues.

59. The diastereomeric peptide of claim 55, wherein said peptide is 5-50 amino acid residues in length.

60. The diastereomeric peptide of claim 55, wherein the TCR transmembrane domain comprises an amino acid sequence as set forth in any one of SEQ ID NOS:4-9.

61. The diastereomeric peptide of claim 55, wherein the TCR transmembrane domain is derived from murine TCR.

62. The diastereomeric peptide of claim 61, the peptide comprising an amino acid sequence as set forth in SEQ ID NO: 1 wherein at least one amino acid residue is of the D-isomer configuration, or derivatives, fragments, analogs, extensions, conjugates and salts thereof.

63. The diastereomeric peptide of claim 62, wherein the diastereomeric peptide has the amino acid sequence GLRILLLKV, wherein the underlined amino acid residues at positions 3 and 8 are of the “D” isomer configuration (SEQ ID NO:2).

64. The diastereomeric peptide of claim 55, wherein the TCR transmembrane domain is derived from human TCR.

65. The diastereomeric peptide of claim 64, wherein said diastereomeric peptide has an amino acid sequence as set forth in SEQ ID NO: 10.

66. The diastereomeric peptide of claim 55, wherein the peptide has an amino acid sequence as set forth in any one of SEQ ID NOS:12-17, 19-28 and 37-47.

67. The diastereomeric peptide of claim 55, wherein the diastereomeric peptide is conjugated to a lipophilic moiety.

68. The diastereomeric peptide of claim 67, wherein the lipophilic moiety is a fatty acid selected from the group consisting of saturated, unsaturated, monounsaturated, polyunsaturated and branched fatty acids.

69. The diastereomeric peptide of claim 68, wherein the fatty acid consists of at least three carbon atoms.

70. The diastereomeric peptide of claim 68, wherein the fatty acid is selected from the group consisting of: octanoic acid (OA), decanoic acid (DA), undecanoic acid (UA), dodecanoic acid (DDA; lauric acid), myristic acid (MA), palmitic acid (PA), stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

71. The diastereomeric peptide of claim 67, wherein the peptide has an amino acid sequence as set forth in any one of SEQ ID NOS:29-36 and 48-50.

72. A peptide derived from a T cell receptor (TCR) alpha chain transmembrane domain, the peptide comprising at least two basic amino acid residues, wherein all amino acid residues of said peptide are of the “D” isomer configuration.

73. A peptide according to claim 72 having an amino acid sequence selected from the group consisting of: GLRILLLKV, (SEQ ID NO:3) and GFRILLLKV, (SEQ ID NO:11), wherein the underlined amino acid residues at positions 1-9 are of the “D” isomer configuration.

74. The peptide of claim 72, which peptide is conjugated to a lipophilic moiety.

75. A pharmaceutical composition comprising as an active ingredient a peptide according to claim 55, and a pharmaceutically acceptable carrier, excipient or diluent.

76. A method of treating a T cell mediated pathology in a subject in need thereof, comprising administering to the subject a therapeutically effective amount of a diastereomeric peptide according to claim 55.

77. The method of claim 76, wherein said diastereomeric peptide has an amino acid sequence as set forth in any one of SEQ ID NOs:1, 2, 10, 12-17, 19-28, 37-47, and derivatives, fragments, analogs, extensions, conjugates and salts thereof.

78. The method of claim 77, wherein the diastereomeric peptide is conjugated to a lipophilic moiety.

79. The method of claim 78, wherein the peptide has an amino acid sequence as set forth in any one of SEQ ID NOS: 29-36 and 48-50.

80. The method of claim 76, wherein the T cell mediated pathology is a T cell-mediated autoimmune disease.

81. The method of claim 80, wherein the autoimmune disease is selected from the group consisting of: multiple sclerosis, autoimmune neuritis, systemic lupus erythematosus (SLE), psoriasis, Type I diabetes (IDDM), Sjogren's disease, thyroid disease, myasthenia gravis, sarcoidosis, autoimmune uveitis, inflammatory bowel disease (Crohn's and ulcerative colitis), autoimmune hepatitis, rheumatoid arthritis, idiopathic thrombocytopenia, scleroderma, alopecia areata, hemolytic anemia, glomerulonephritis, dermatitis and pemphigus.

82. The method of claim 81, wherein the autoimmune disease is rheumatoid arthritis.

83. The method of claim 76, wherein the T cell mediated pathology is a T cell-mediated inflammatory disease.

84. The method of claim 76, wherein the T cell mediated pathology is selected from the group consisting of: allograft rejection and graft-versus-host disease.

85. A method of inhibiting T-cell activation in a subject in need thereof, comprising administering to the subject a therapeutically effective amount of a diastereomeric peptide according to claim 55.

86. The method of claim 85, wherein at least two amino acid residues of the diastereomeric peptide are of the D-isomer configuration, and wherein the peptide is 5-50 amino acid residues in length.

87. The method of claim 85, wherein said diastereomeric peptide has an amino acid sequence as set forth in any one of SEQ ID NOs:1, 2, 10, 12-17, 19-28, 37-47, and derivatives, fragments, analogs, extensions, conjugates and salts thereof.

88. The method of claim 85, wherein the diastereomeric peptide is conjugated to a lipophilic moiety.

89. The method of claim 88, wherein the peptide has an amino acid sequence as set forth in any one of SEQ ID NOS:29-36 and 48-50.

90. A method of treating a T cell mediated pathology or inhibiting T-cell activation in a subject in need thereof, comprising administering to the subject a therapeutically effective amount of a peptide according to claim 72 or a lipophilic conjugate thereof.

91. A pharmaceutical composition comprising as an active ingredient a peptide according to claim 72, and a pharmaceutically acceptable carrier, excipient or diluent.
